(19) United States
(12) Patent Application Publication
(10) Pub. No.: US 2022/0364998 A1
(43) Pub. Date: Nov. 17, 2022

(54) SOIL WATER COLLECTION AND ANALYSIS SYSTEMS AND RELATED METHODS

(71) Applicant: Precision Planting LLC, Tremont, IL (US)

(72) Inventors: Dale M. Koch, Tremont, IL (US); Nicholas Minarich, Shorewood, IL (US); Todd Swanson, Morton, IL (US); Rachel Nelson, Tremont, IL (US); Adam Vaccari, Tremont, IL (US)

(21) Appl. No.: 17/754,984

(22) PCT Filed: Sep. 28, 2020
(86) PCT No.: PCT/IB2020/059043

§ 371 Date: Apr. 18, 2022

Related U.S. Application Data
(60) Provisional application No. 62/934,049, filed on Nov. 12, 2019.
(60) Provisional application No. 62/916,180, filed on Oct. 16, 2019.

(51) Int. Cl. G01N 21/78; G01N 21/94; G01N 35/00; G01N 33/24; G01N 1/40

(52) U.S. Cl. CPC G01N 21/78; G01N 21/94; G01N 35/00009; G01N 33/24; G01N 1/4077; G01N 2035/00019; G01N 2033/245; G01N 2001/4088

(57) ABSTRACT

A system for collecting and chemically analyzing water samples extracted from the soil to measure one or more analytes of interest such as soil nutrient levels of agricultural interest for increasing crop yield and quality in one use of the system. The system includes a sample collection probe (20) comprising a filter media (26) arranged to contact the soil when embedded therein and capture a water sample from the soil, and operably coupled to a sample processing sub-system (180) thereby collectively forming a sampling station (190). The sub-system (180) is configured to receive and analyze the water sample. A programmable probe controller (60) directs operation of the sample collection, processing, and chemical analysis in situ. A networked array (110) of sampling probes dispersed throughout the field may communicate wirelessly with at least one remote electronic device such as via the cloud computing (102). A modular version of a sampling probe (200) permits customized sampling at various soil depths.

### BACKGROUND

The present disclosure relates generally to systems for soil analysis, and more particularly to such systems and related devices and methods for performing quantitative soil analyses.

Soil analysis and testing is an important aspect of the agricultural arts. Test results provide valuable information on the chemical makeup and characteristics of the agricultural soil (e.g. levels of nitrogen, phosphorous, potassium, etc.) so that various amendments may be added to the soil to maximize the quality and quantity of crop production. Samples are generally processed to quantify the presence of particular analytes (i.e. substances or chemical constituent of interest), which can then be used to ascertain the amounts and types of soil amendments and fertilizers required for various areas of the agricultural field.

Improvements in soil testing and analysis are desired.

### BRIEF SUMMARY

The present disclosure provides a system and related devices and methods/processes for collecting and analyzing soil pore water samples to determine plant nutrient or other parameter levels/concentrations in the soil which may be relevant for agricultural purposes in one non-limiting application. The soil water collection and analysis system may incorporate the soil pore water extraction/collection, processing, and chemical analysis functions into a single unified equipment platform which is portable and may be remotely deployed in the agricultural field. The soil water collection and analysis system operates by capturing or extracting a soil water sample, which will contain the particular analytes of interest (e.g. nitrogen, potassium, or other), combining the sample with one or more reagents specifically selected to cause a chemical reaction with the targeted analyte(s), and analyzing the resultant property change in the water-reagent mixture (e.g. color, pH, or other aspect/parameter) from the interaction between the reagent and analyte present in the collected sample which will be indicative of the level of analyte present. Any suitable analytical technique and device may be used to determine the level or amount of analyte present. Non-limiting examples include colorimetry, Ion Selective Electrodes (ISE's), and Ion exchange Resins. The system may be configured and operable to analyze any variety of analytes or parameters of the soil (e.g. levels of nitrogen, phosphorous, potassium, pH, temperature, etc.).

In various embodiments, some of the component parts of the system for collecting the water samples may be configured for semi-permanent or permanent stationary and direct implantation into the soil of the agricultural field, or alternatively may be a part of a mobile system mounted to a moving agricultural vehicle (e.g. towed, pushed, or self-propelled). For stationary implantation in soil, a lysimeter-type soil water sample collection probe (alternatively “sample probe” for short as also used herein) is provided. The sample probe may have an elongated generally tubular body comprising a filter media for direct contact with the soil and interior cavity for collecting the filtered water sample (filtrate). The probe may have a rounded, cone-shaped, or other readily implantable shaped end in some embodiments.

The sample probe is fluidly and operably coupled to the sample processing sub-system portion of the soil water collection and analysis system. The sample processing sub-system may be housed in an in situ equipment enclosure which contains the active devices, electronics, fluid components, and others needed for processing and chemically analyzing the collected filtrate from the sample probe for determining the levels of soil nutrients or other relevant parameters (e.g. pH, etc.). The equipment enclosure may be closely coupled to the probe in the field in close proximity, such as adjacent thereto and/or physically coupled to the probe at the soil sampling site. In such an implementation, the combination of sample probe and sample processing sub-system may therefore define and be configured to serve as a remote fully functional soil water analysis lab or station. The sampling station is operable to both collect and fully analyze the soil water sample from the agricultural field. Optionally, the sampling station may communicate the collected data and analysis results externally to a remote programmable processor-based central control system which acts as a repository for soil analysis data and storage. This soil water sampling station or unit may contain a probe controller comprising one or more programmable microprocessors. The controller may be configured via appropriate software or program instructions to control the entire operation of sampling station. The probe controller may include a communications interface which is communicably linked (e.g. wirelessly) to one or more remote electronic devices for data sharing and/or control signal interchange. Accordingly, the soil water collection and analysis system embodied in such a sampling station may be fully automated for collecting and analyzing the soil moisture sample to detect the presence and level of an analyte.

In some embodiments, a sampling network comprising a plurality of sampling stations each including the combination of a soil water sample collection probe and a sample processing sub-system may be implanted in various spatially-separated locations throughout the agricultural field. Each sampling station may be may be communicably linked via the cloud (i.e. cloud computing) or other wireless communication means to the one or more remote electronic devices; at least one of which may serve as a processor-based central control system to exchange data and control signals with the sampling station. In some embodiments, the central control system device which becomes a central or main controller may be configured to direct and control the operation of these remote sampling stations. Each sampling station may be configured to analyze the same or different analytes (e.g. nitrogen) which the central control system may use to generate both a moisture and chemical profile of various regions of the agricultural field indicating specific nutrient levels for each. This information allows the end user (e.g. farmer) to know which areas are deficient in various soil nutrients so that application of suitable types and amounts of soil amendments/fertilizers can be better planned in advance. The control system may be configured to simultaneously process and analyze data received from a plurality of soil water sampling stations in parallel in real-time, thereby saving analysis time and allowing the user to see a snapshot of all soil nutrient levels at once.

In some embodiments, the sample probe may be a modular type sampling probe system comprising one or more extension members and one or more sampling modules each which include a filter media. The sampling modules and extension members may be detachably coupled together to form various configurations and lengths of the modular sampling probe for collecting soil water samples at a single predetermined soil depth, or at several different depths using the same modular unit. The modular system may include anti-rotational couplings at the joints between different sections and appurtenances of the modular sample probe. In one embodiment, the anti-rotational couplings may comprise splined interfaces formed by complementary configured male and female splined couplings which provide a torque resistant jointed assembly for rotatably implanting the probe into the soil that overcomes resistance imparted to the probe by the soil. The modular probe sampling system advantageously allows the user or manufacturer to easily customize the water collection depth(s) in the soil using a plurality of interchangeable and detachably coupled components preferably sharing a common anti-rotational mounting interface.

Although the present soil water analysis system may be described herein with reference to quantification of plant nutrient and related parameters of the soil, the system is expressly not limited in its applicability to agricultural-related uses alone. Accordingly, the soil water analysis system may be readily adapted for use to quantify other type chemical characteristics (e.g. heavy metals, arsenic, lead, etc.) which are relevant to a variety of groundwater and soil pore water routine monitoring applications for soil contaminants in industries such as mining, petroleum, power generation plant waste basin monitoring, landfills, and others.

In one aspect, a system for collecting and analyzing soil water samples comprises: a sample probe comprising a filter media arranged to contact the soil when embedded therein, the sample probe configured for collecting a water sample from the soil; and a sample processing sub-system located proximately to the sample probe, the processing system operably coupled to the sample probe and configured to extract and analyze the water sample for at least one analyte. The sample processing sub-system may include processor-based programmable probe controller; the probe controller configured to direct operation of the sub-system.

According to another aspect, a modular sampling probe for collecting soil water samples comprises: at least one sampling module comprising a filter arranged to contact the soil when embedded therein, the sampling module configured and operable to extract and filter a water sample from the soil; and at least one extension member coupled to the at least one sampling module at a first joint. The first joint may be an anti-rotational splined joint comprising a male splined coupling on the at least one extension member or the at least one sampling module, and a female splined coupling on a remaining other one of the at least one extension member or the at least one sampling module.

A method for assembling and using the modular soil sampling probe comprises: providing the at least one sampling module which is a first sampling module; and coupling the first sampling module to the at least one extension member which is a first extension member, the first sampling module and first extension member collectively forming the sampling probe. The method may further comprise coupling a second sampling module to the first extension member, the second sampling module being the same as the first sampling module. The method may further comprise coupling the first sampling module to a second extension member. The step of coupling the first sampling module to the first extension member may further comprise slideably inserting male spline protrusions disposed the first sampling module or first extension member into female spline channels disposed on the other one of the first sampling module or first extension member. The first sampling module may be detachably coupled to the first extension member by a first coupling member having a first set of splines slideably engaging a mating first set of spline channels formed on the first extension member.

According to another aspect, a system for collecting and analyzing a soil water sample comprises: a sampling probe configured for embedment in soil; at least one wicking member including a first portion arranged to contact the soil and capture a water sample, and a second portion inside the probe, the wicking member formed of a wicking material structured to extract and transport the captured water from the soil via capillary action; and a water sample analysis device configured to measure an analyte present in the water sample captured by the wicking member.

According to another aspect, a system for collecting and analyzing a soil water sample comprises: a sampling probe configured for embedment in soil; a sampling window disposed on sampling probe and in direct contact with the soil outside of the sampling probe; and a water sample analysis device configured to emit ultraviolet light at the soil through the sampling window and measure reflectance of the ultraviolet light from the soil to measure an analyte present in pore water of the soil.

According to another aspect, a system for collecting and analyzing a soil water sample comprises: a sampling probe configured for embedment in soil; a rotatable carousel disposed inside the sampling probe, the carousel comprising a plurality of circumferentially arranged color-indicating patches; a sample collection device configured and arranged to collect the water sample from the soil and wet the color-indicating patches; wherein the patches are operable to change color in the presence of an analyte in the soil water sample.

### BRIEF DESCRIPTION OF THE DRAWINGS

The present disclosure will become more fully understood from the detailed description and the accompanying drawings, wherein like elements are labeled similarly and in which:

FIG. 1 is a schematic diagram of a soil water sampling system according to one embodiment of the present disclosure comprising a water sampling probe and associated sample processing sub-system including a first embodiment of a vacuum device for applying negative pressure to the probe;

FIG. 2 is a side view of the sampling probe embedded in soil for capturing pore water;

FIG. 3 is a side view showing a second embodiment of a vacuum device for applying negative pressure to the probe;

FIG. 4 is schematic diagram of a cloud-based networked communication system for sharing soil water analysis data and interchanging command signals to operate portions of the sampling system;

FIG. 5 is a side view of a first embodiment of a method for embedding the sample probe in the soil and establishing intimate contact between the probe and soil for extracting a water sample;

FIG. 6 is a side view of a second embodiment of a method for embedding the sample probe in the soil and establishing intimate contact between the probe and soil for extracting a water sample;

FIG. 7 is a side view of a third embodiment of a method for embedding the sample probe in the soil and establishing intimate contact between the probe and soil for extracting a water sample;

FIG. 8 is a side view of a fourth embodiment of a method for embedding the sample probe in the soil and establishing intimate contact between the probe and soil for extracting a water sample;

FIG. 9 is a side view of a sample probe with external threads;

FIG. 10 is a first side view of a hydrophilic type sampling probe in the form of a testing cassette according to the present disclosure;

FIG. 11 is a perspective view thereof;

FIG. 12 is a second side cross sectional view thereof;

FIG. 13 is a side view of a portion of the reel of cassette tape with test pad thereon of the sampling probe of FIG. 10;

FIG. 14 is a side view of a multi-layer composite block or test pad on the cassette tape of the sampling probe of FIG. 10;

FIG. 15 shows a mechanical method for liberating captured water for a saturated test pad of the sampling probe of FIG. 10;

FIG. 16 shows a chemical method for liberating captured water for a saturated test pad of the sampling probe of FIG. 10;

FIG. 17 is a first perspective view of a modular sampling probe system comprising a modular sampling probe assembly for capturing soil water;

FIG. 18 is a second perspective view thereof;

FIG. 19 is an exploded view thereof;

FIG. 20 is a first longitudinal side view thereof;

FIG. 21 is a second longitudinal side view thereof;

FIG. 22 is a third longitudinal side view thereof;

FIG. 23 is a longitudinal side cross sectional view thereof;

FIG. 24 is a detailed view taken from FIG. 23;

FIG. 25 is a first transverse cross-sectional view taken from FIG. 24;

FIG. 26 is a second transverse cross-sectional view taken from FIG. 24;

FIG. 27 is an enlarged longitudinal cross sectional view of one end of the modular sampling probe showing internal fluid passages;

FIG. 28 is a third transverse cross sectional view taken from FIG. 24;

FIG. 29 is cross-sectional perspective view of a coupling member of the modular sampling probe;

FIG. 30 is an alternative enlarged cross-sectional perspective view of one end thereof;

FIG. 31 is a transverse cross section taken from FIG. 29;

FIG. 32 is an enlarged detail taken from the longitudinal probe cross section of FIG. 37 showing coupling of a conical end closure to the sampling probe;

FIG. 33 is an end perspective view of the probe showing the end closure;

FIG. 34 is a perspective view of the modular sampling probe showing the end closure and a flow manifold housing coupled to the probe;

FIG. 35 is a first side view thereof;

FIG. 36 is a second side view thereof;

FIG. 37 is a longitudinal cross-sectional view thereof;

FIG. 38 is a transverse cross-sectional view of the flow manifold housing;

FIG. 39 is a transverse cross sectional view taken from FIG. 37 showing coupling details between the flow manifold housing and the probe;

FIG. 40 is an enlarged detail taken from FIG. 37;

FIG. 41 is a side longitudinal cross sectional view showing coupling details between the end closure and a sampling module;

FIG. 42 is first perspective view of the flow manifold housing;

FIG. 43 is a second perspective view of the flow manifold housing showing an internally splined coupling detail;

FIG. 44 is a first perspective view of the conical end closure;

FIG. 45 is a second perspective view thereof showing a first type of splined coupling end;

FIG. 46 is a third perspective view thereof showing a second type of splined coupling end;

FIG. 47 shows an alternative embodiment of the powered sampling tape drive of testing cassette of FIG. 10 configured for use with a sampling tape which includes a removably protective overlay film;

FIG. 48 shows a first embodiment of a soil sampling station with self-contained water source for obtaining a soil sample;

FIG. 49 shows a second embodiment of a soil sampling station with self-contained water source for obtaining a soil sample; and

FIG. 50 shows a third embodiment of a soil sampling station with self-contained water source for obtaining a soil sample.

All drawings are schematic and not necessarily to scale. Components numbered and appearing in one figure but appearing un-numbered in other figures are the same unless expressly noted otherwise. A reference herein to a whole figure number which appears in multiple figures bearing the same whole number but with different alphabetical suffixes shall be constructed as a general refer to all of those figures unless expressly noted otherwise.

### DETAILED DESCRIPTION

The features and benefits of the invention are illustrated and described herein by reference to exemplary (“example”) embodiments. This description of exemplary embodiments is intended to be read in connection with the accompanying drawings, which are to be considered part of the entire written description. Accordingly, the disclosure expressly should not be limited to such exemplary embodiments illustrating some possible non-limiting combination of features that may exist alone or in other combinations of features.

In the description of embodiments disclosed herein, any reference to direction or orientation is merely intended for convenience of description and is not intended in any way to limit the scope of the present invention. Relative terms such as “lower,” “upper,” “horizontal,” “vertical,”, “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise.

As used throughout, any ranges disclosed herein are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

### Vacuum Type Sampling System

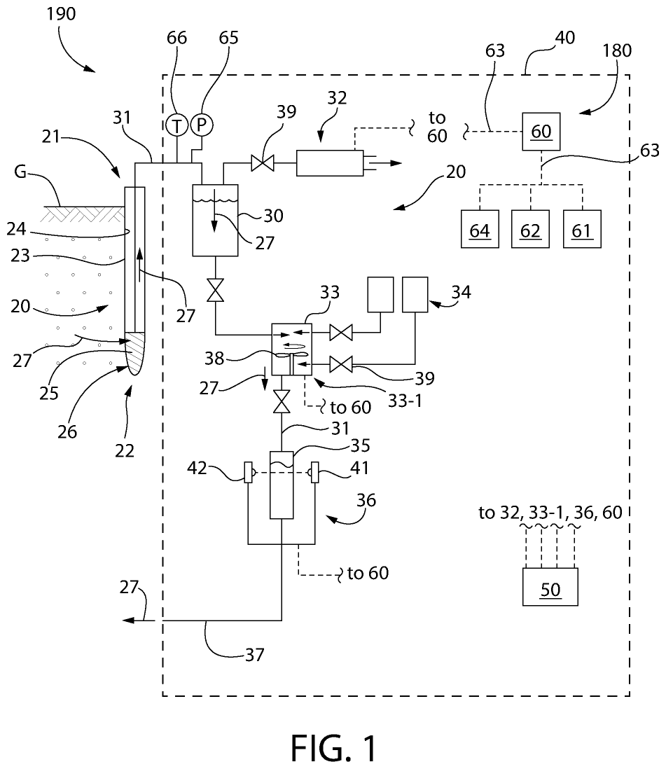

FIG. 1 is a schematic flow diagram of a soil water analysis system comprising a first embodiment of a soil water sample collection lysimeter or probe 20 and an associated closely coupled sample processing sub-system 180. It bears noting that the sample processing sub-system 180 is mounted in the agriculture field proximate and preferably adjacent to the probe 20 which captures the water sample. This combination advantageously defines and provides a complete soil water analysis sampling station 190 with capabilities to extract, process, and analyze the water sample in situ and in real-time to quantify an analyte of interest (e.g. soil nutrients). The system and its components are further described in turn below.

Sample probe 20 may have an elongated tubular housing or body in one non-limiting embodiment including a probe longitudinal axis LA, a top end 21, bottom end 22, and circumferentially-extending sidewall 23 extending axially between the ends. The sidewall may have an at least partially solid structure for at least part of its length, or a majority of its length in some constructions. The top end 21 may be closed and sealed except for passage of a flow conduit therethrough as further described herein. The body may be formed of any suitable metallic or non-metallic material including polymeric materials (e.g. PVC, polyethylene, etc.) and preferably corrosion resistance metals if used (e.g. aluminum or stainless steel). The probe body is configured for implantation in the soil S beneath the ground surface G.

An internal cavity 24 extends axially between the top and bottom ends 21, 22 of the probe for collection water from the adjacent soil. The probe sidewall 23 which defines the cavity may be cylindrical in one non-limiting embodiment forming a circular transverse cross-sectional shape. In other possible embodiments, however, sidewall may have other non-polygonal or polygonal cross-sectional shapes (e.g. square, rectangular, hexagonal, octagonal, etc.). The bottom end 22 of probe 20 may have any suitable shape. In some embodiments, bottom end 20 may be simply flat or straight (i.e. perpendicular to probe longitudinal axis LA) as shown in FIG. 24. In other embodiment, bottom end 22 may be shaped to facilitate insertion into the soil, such as for example rounded as shown, conical or spike shaped, or otherwise.

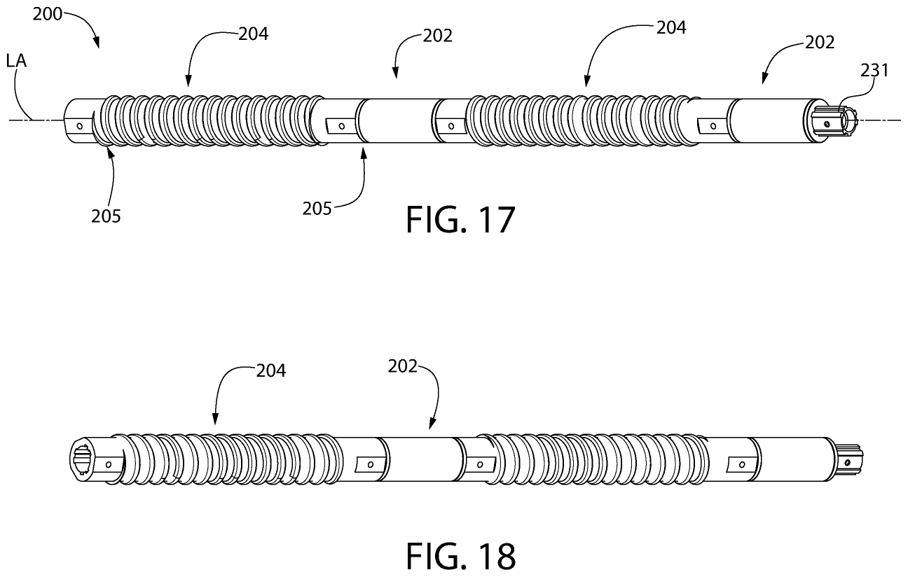

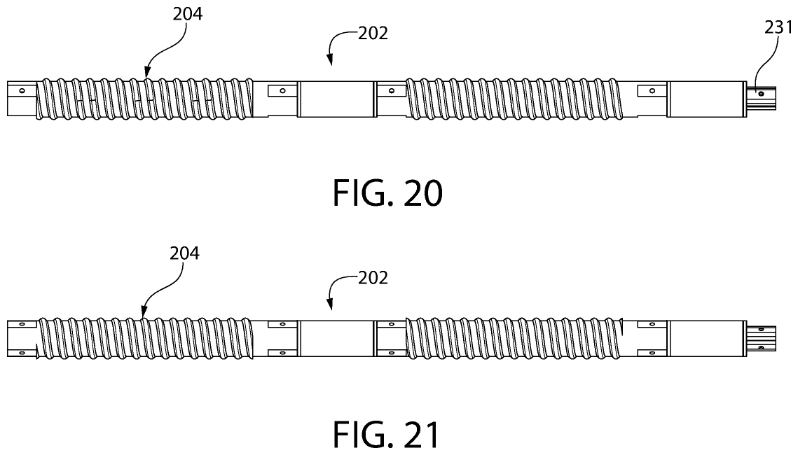

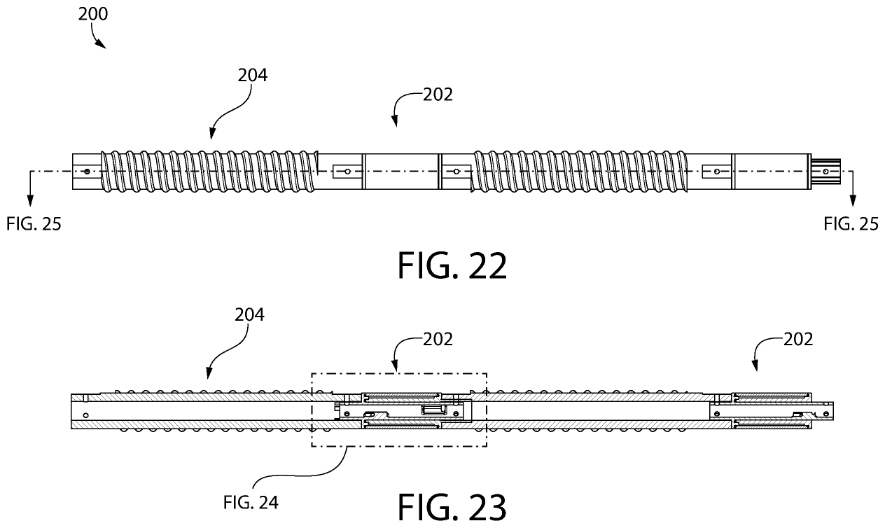

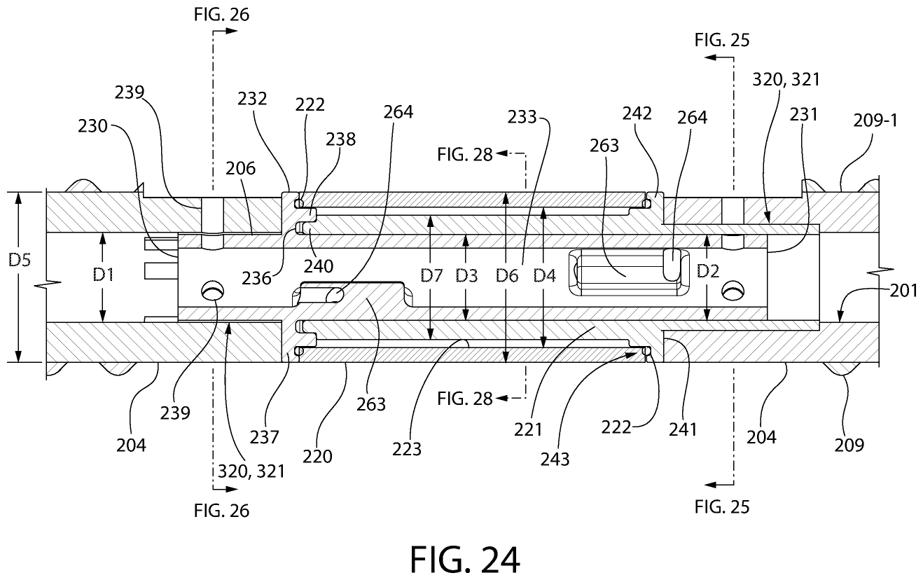

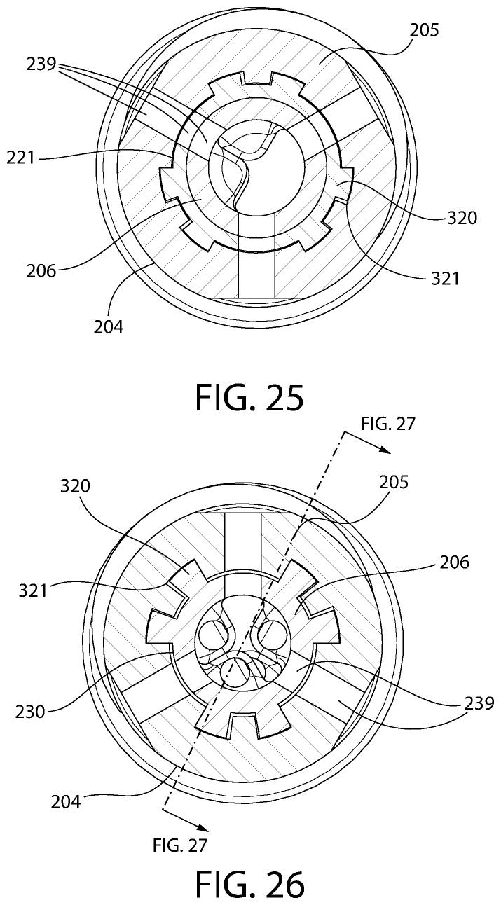

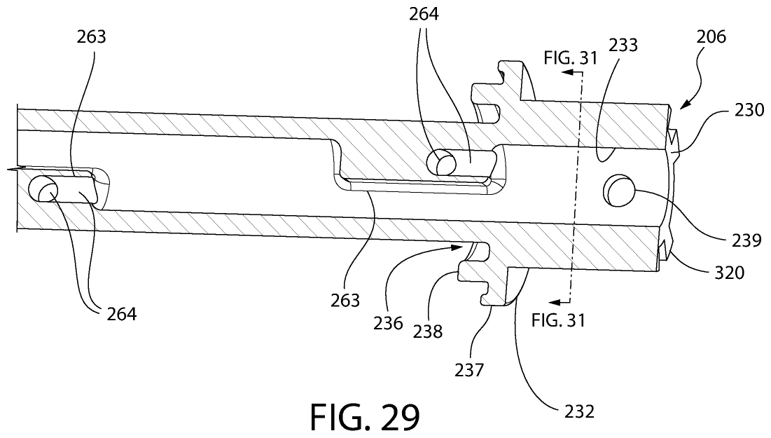

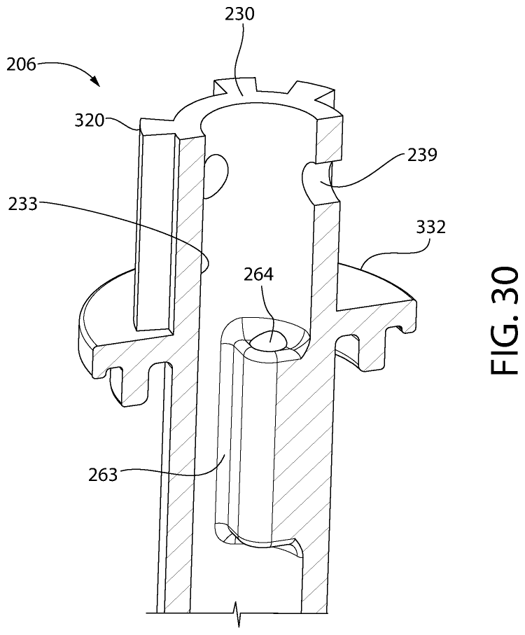

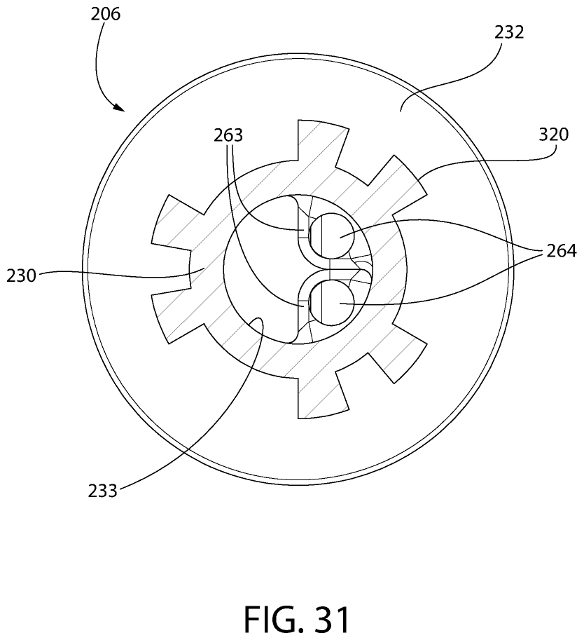

Probe 20 further includes a porous filter or media 26 configured for drawing water from the soil into the interior of the probe for collection and analysis. In one embodiment, the porous media may comprise a porous tip 25 mounted to the bottom end 22 of the probe. The tip 25 may extend upwards from the bottom end for a distance along the sides of the probe as shown. In other embodiments shown in FIGS. 17-30, the porous media 26 in the form of a porous filter 220 may form partial or complete circumferential cylindrical segments forming a portion of the sidewall 25 of probe 20, as further described herein. In such embodiments, the bottom end 22 of probe 20 may have a solid non-porous flat, conical, or other shaped structure.

The porous media may be formed of any suitable material having a suitable pore or opening size or selected to preclude drawing particles of soil into the probe, but allow water to flow through the media. Suitable porous filter or media includes for example without limitation porous ceramic, sintered metal, polymers, polymeric or fibrous membranes, screens, etc.). The invention is not limited by the type of porous media used. In other possible embodiments, the porous media may comprise affixing the porous media to an open section or multiple open sections of the sidewall 23 of the probe between the top and bottom ends 21, 22 to draw water laterally into the probe.

The soil water sample processing sub-system 180 associated with sample probe 20 further includes collection chamber 30 which receives the collected soil water sample, mixer 33-1 which comprises a mixing chamber 33, one or more reagent chambers 34, sample analysis chamber 35, vacuum pump 32, and a soil water sample analysis device 36 for measuring the concentration/level of analyte in the water sample. The porous media 26, collection chamber 30, mixing chamber 33, analysis chamber 35, and reagent chamber(s) 34 may be fluidly coupled together via suitable flow conduits 31 such as tubing (not all tubing has been numbered in FIG. 1 for clarity of depiction). Any suitable rigid or flexible non-metallic or metallic tubing material may be used such as polyethylene, PTFE (polytetrafluoroethylene), etc. Collection chamber 30 is fluidly coupled to the porous media 26 and mixing chamber 33. Each reagent chamber is fluidly coupled to the mixing chamber. The analysis chamber 35 which is in turn fluidly coupled to the mixing chamber. One or more valves 39 may be used to control and time the flow of water and reagents to the mixing chamber 38.

Mixing chamber 38 includes an electric motor driven mixing blade assembly 38. The blade assembly operates to mix the soil water sample and reagents to induce a chemical reaction which can be detected and the analyte concentration thus measured by the sample analysis device. In other embodiments contemplated, the “mixing chamber” could also be any apparatus or method of mixing or moving solution around, such as a magnetic stir bar, circulation pump, shaker, or just plain perturbed.

Vacuum pump 32 may be electric motor driven and is fluidly coupled to the mixing chamber 38 via a flow conduit 31. Pump 32 is configured and operable to draw a vacuum through the porous media 26 for pulling water from the surrounding soil into the probe. The pump then draws the water sample into the mixing chamber from the media via a flow conduit 31 extending through the probe body, as shown. Any suitable commercially-available vacuum pump may be used for this application.

A suitable electric power source 50 may be provided and electrically coupled to the vacuum pump 32, mixing chamber 38, sample analysis device 36, and other components requiring power associated with probe 20. Power source 50 may be a rechargeable battery unit for remote placement of the sample probes in the agricultural field. In some embodiments, a rechargeable solar batter unit including solar panel or sensor for recharging the batteries via sunlight may be used. This may be particularly advantageous considering the remote placement of the probe or probes in the field.

In one embodiment, the sample analysis device 36 may be an electronic device comprising an absorbance measurement colorimeter which operates at a specific wavelength to detect the particular analyte of interest, such as for example without limitation about 525 nm for detection of a nitrate indicator or 210 nm for detection of nitrate itself, or others. It will be appreciated that in this example, colorimetric analysis and detection of nitrate at 525 nm or 210 nm is only for a specific nitrate reduction reaction. Other analytes may require device 36 to operate at other wavelengths specific to detection of such other analytes. Sample analysis device 36 operably cooperates with analysis chamber 35 which may be a transparent vessels placed between the LED (light emitting diode) transmitting diode 41 and LED receiving diode 42. As the reacted water and reagent mixture flows past the device, the absorbance of particular wavelengths of light by the mixture is measured to quantify the concentration of the analyte in the water sample. Such colorimeters are commercially-available.

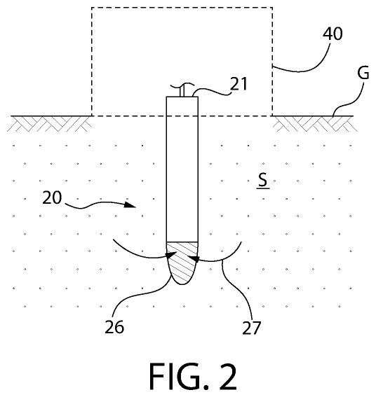

The collection chamber 30, mixing chamber 33, one or more reagent chambers 34, sample analysis chamber 35, vacuum pump 32, a soil water sample analysis device 36, power source 50, and other related appurtenances including valving and tubing may be mounted in a separate equipment enclosure 40 (schematically designated by the dashed box in FIG. 1) from the probe 20. The enclosure may have any suitable configuration for the probe installation site. In various constructions, the equipment enclosure may be mounted directly above or on top of the probe (see, e.g. FIG. 2), or in another other position adjacent to the probe. In some embodiments, the top end 21 of the probe 20 may be positioned inside the enclosure as also shown in FIG. 2.

A method or process for collecting and analyzing a soil water sample using the system of FIG. 1 will now be briefly described. First, the sample probe 20 is implanted into the soil to be tested such that the porous media 26 is in intimate contact with the surrounding soil at the desired depth for collecting the water sample. A vacuum is then drawn by operating the vacuum pump 32. The water sample (see directional water flow arrows 27) is drawn transversely/horizontal through the porous media 26 into probe cavity 24, and then vertically upward from the bottom to top of the probe 24 parallel to the longitudinal axis LA. In other possible filter media arrangements, water could be drawn through the media in other directions or orientations (e.g. vertically or obliquely to axis LA). The collected water is then deposited into mixing chamber 30 via flow conduit 31. Operation of vacuum pump 32 is then stopped.

The extracted soil water sample next flows downward via gravity to mixing chamber 38 from collection chamber 30 by opening previously closed mixing chamber inlet valve 39. In alternative embodiments contemplated, collection chamber 30 may be omitted and the extracted soil water sample may instead be deposited directly into mixing chamber 38. Next, the reagent chambers 34 are then fluidly connected to the mixing chamber by opening the reagent valves 39 to introduce reagent into the sample. The mixing chamber inlet valve and reagent valves are then closed. The mixer is operated so that blade assembly 38 thoroughly mixes the reagent and water sample. This causes a chemical reaction which changes the color of the resulting water-reagent mixture. The previously closed mixing chamber outlet valve 39 is then opened which causes the water-reagent mixture to flow through the clear analysis chamber and outwards from equipment enclosure 40 through drain line 37. The mixing chamber inlet valve may optionally be opened if needed to break any vacuum created in the mixing chamber 33. As the water sample flows through the colorimeter (sample analysis device 36), the analyte (e.g. nitrate or other) is measured to determine its concentration in the sample which is indicative of the concentration or level of the analyte in the surrounding soil.

It bears noting that the volume of reagent held in the reagent chambers 34 may be more than necessary to process a single soil water sample. Accordingly, a single reagent charge or fill of the reagent chambers may be capable of processing multiple samples. In such an implementation, the reagent valves may be operated to dose the water sample in the mixing chamber 33 with only the amount necessary each time to cause a chemical reaction with the analyte for analysis.

In some embodiments, the foregoing sampling and analysis process may be fully automated by providing a “smart” probe comprising a probe control system comprising a programmable probe controller 60 operably and communicably coupled to the foregoing components of the sample processing sub-system 180 via wireless and/or wired communication links 63 to control their operation and operating sequence in the manner described above. Controller 60 thus provides a user-configurable control system for the probe sampling system. Individual communication links to each component (e.g. valves, pump, chambers, mixer, sample analysis device, etc. which are not shown in FIG. 1 to avoid undue clutter). The controller 60 based control system may also be housed inside the equipment enclosure 40 which may be at least partially sealed from the weather and environment to protect the electronics.

The programmable probe controller 60 includes a programmable processor which may be one or more processors or microprocessors, a system on a chip (integrated circuit), one or more microcontrollers, ASICs (application specific integrated circuits), or combinations thereof. Probe controller 60 includes processing logic for executing programmable control logic or software instructions comprising one or more programs which are executed by the controller to control operation of the sampling system.

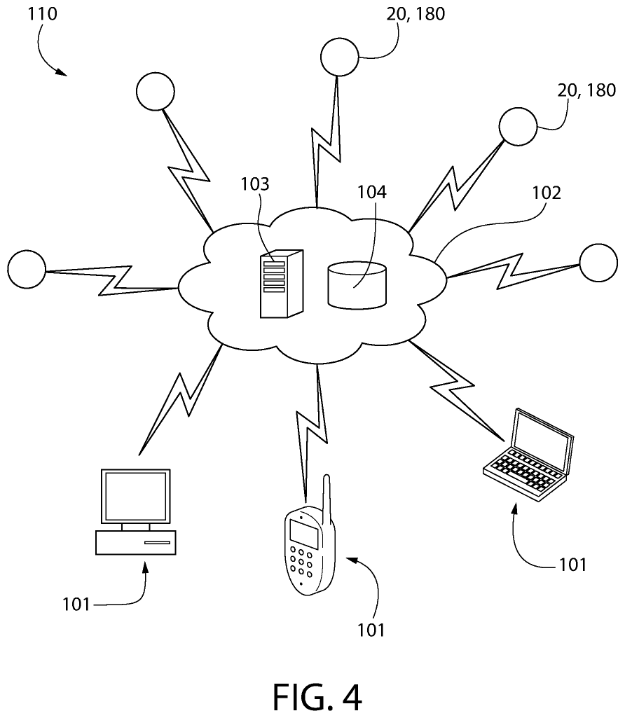

Probe controller may further include an input/output communication interface or module 61 configured for wireless and/or wired communication for programming the processor and exchanging sampling results or other data via external processor-based personal electronic devices (e.g. computer, cell phone, tablet, laptop, etc.). Communication module 61 therefore may be configured for transmitting and receiving communications and data via wireless and/or wired protocols to and from one or more user's personal electronic devices. This allows the user or users to be operably coupled to the probe controller 60 for uploading software to configure and control operation of the probe controller 60, and for downloading soil water sampling related measurement, analysis, and other data or results. In some embodiments, the user's personal electronic device 101 may therefore act as processor-based main control system by running appropriately configured software, as further described elsewhere herein. The personal electronic devices 101 may be communicably coupled or linked to probe controller 60 either directly (e.g. Wi-Fi, near field communication (NRC), Bluetooth®, coupled wired connection or jack, etc.), or alternatively via the Internet such as by cloud computing in which both the personal electronic device and probe controller 60 are in turn communicably linked via the “cloud” 102, as further described herein (see, e.g. FIG. 4).

Probe controller 60 further includes non-transitory tangible computer readable medium 62 operably coupled and accessible to the controller 60. The computer or machine accessible and readable medium may include any suitable volatile memory and non-volatile memory or devices operably and communicably coupled to the controller processor(s). Any combination and types of volatile or non-volatile memory may be used including as examples, without limitation, random access memory (RANI) and various types thereof (e.g. ferroelectric RANI, DRAM, etc.), read-only memory (ROM) and various types thereof, hard disk drives (HDD), solid-state drives (SSD), flash memory, SD card, USB drive, or other suitable memory and devices which may be written to and/or read by the processor operably connected to the medium. The non-volatile memory may thus be any permanent or removable type memory. Both the volatile memory and the non-volatile memory are used for saving data or results from processed samples, for storing programming (program instructions or software), and storing operating parameters associated with operation of the rotary machine 101 or processing samples, etc. Both the volatile memory and the non-volatile memory may be used for storing the program instructions or software.

The computer or machine accessible and readable non-transitory medium 62 (e.g. memory) contains executable computer program or software instructions which when executed by the probe controller 60 cause the system to perform operations or methods of the present disclosure including measuring properties and testing of soil water sample. While the machine accessible and readable non-transitory medium 62 (e.g., memory) is shown in an exemplary embodiment to be a single medium, the term should be taken to include a single medium or multiple media (e.g., a centralized or distributed database, and/or associated caches and servers) that store the one or more sets of control logic or instructions. It is well within the ambit of one skilled in the art to provide and configure a controller with all the required appurtenances to provide a fully functional control system for operating the probe sampling system and processing soil water samples in the manner disclosed herein.

The probe controller 60 may further include a GPS module 64 which allows the controller to know its precise geo-coordinates where implanted in the agriculture field and transmit its location to the central control system 100 embodied by the user's personal electronic device. The control system 100 may thus act as a hub which keeps track of the locations of a plurality of probes 20 deployed in the agriculture field, as further described herein.

It bears noting that the probe control system includes all other usual appurtenances and ancillary devices known in the art necessary to form a fully functional and programmable probe control system.

In some embodiments, one or more temperatures sensors 66 and pressure sensors 65 may be provided which are communicably coupled to the programmable probe controller 60. The temperature sensor 66 is arranged to measure a real-time actual temperature of the collected soil sample and transmit the measured temperature to the controller. The temperature sensor 66 may be located at any suitable point in the system to measure the water temperature. In one non-limiting example, the temperature sensor 66 may be arranged to measure the temperature of the collected water in the inlet flow conduit 31 to the collection chamber 30. Any suitable type commercially-available temperature sensor may be used (e.g. thermistor, RTD, etc.).

The pressure sensor(s) 65 is arranged to measure pressure of the collected water sample extracted from the soil via probe 20 under negative pressure (vacuum) created by the vacuum pump 32. Pressure sensor 65 is preferably located either in the probe or inlet flow conduit 31 upstream of the collection chamber 30. Any suitable commercially-available pressure sensor may be used.

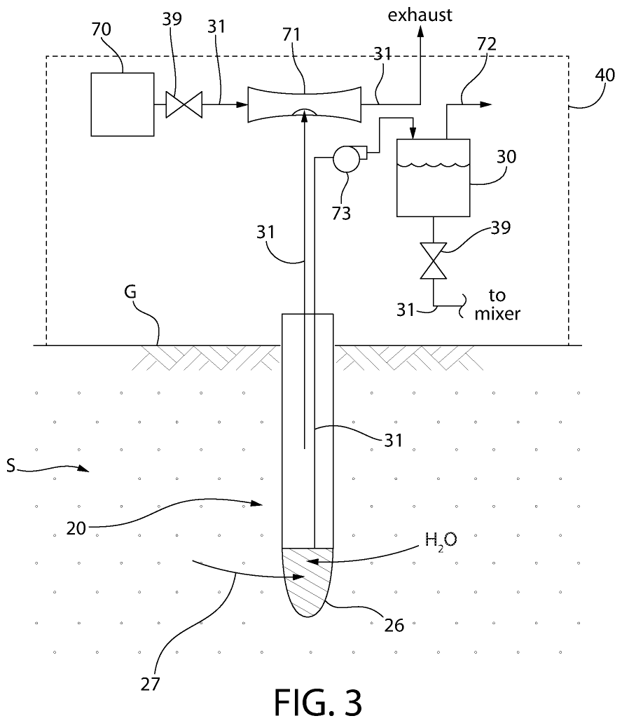

FIG. 3 shows an energy conservation version of the basic system of FIG. 1 which prolongs onboard battery life of the probe system. In this energy conserving system, the vacuum pump is replaced by a combination of an added small water pump 73 with low power consumption requirements and an air-powered venturi jet pump 71. The jet pump is only run for short time sufficient to create an initial negative pressure or vacuum inside the probe to start the inflow of water through the porous media 26 at the start of a sampling cycle. Operation of the jet pump is then terminated. A high pressure air source 70, which may be a canister or tank of pressurized air, is fluidly coupled to an air inlet port of the venturi jet pump 71 via an inlet flow conduit 31. In some embodiments, the air tank may be mounted on, within, or adjacent to the equipment enclosure 40. A vacuum flow conduit 31 is fluidly coupled to the internal cavity 24 of probe 26 media 26 and an inlet to the jet pump 71 to draw a vacuum on the porous media. A separate water inlet flow conduit 31 is fluidly coupled to the porous media 26 and suction or inlet of the water pump 73.

In operation, the air discharge valve 39 fluidly coupled to the air tank is opened which establishes a flow of high pressure air through the jet pump 71. The motive force of the high pressure air flowing through the narrowed throat section of the jet pump with venturi shape draws a vacuum on the probe internal cavity 24, which induces an inflow of water from the surrounding soil inwards through the porous medium 26 into the probe cavity. Air discharge valve 39 is then closed. Next, water pump 73 may be started to draw and pump the extracted water from probe 20 into collection chamber 30. The pump may then be stopped and water is transferred via gravity to the mixing chamber 33 of mixer 33-1 for addition of reagent, mixing, and further processing through the system of FIG. 1 for analysis. The remainder of the sample analysis system and related analysis process is the same as previously described herein with respect to FIG. 1. Any suitable commercially-available venture jet pump may be used for this application.

In other embodiments contemplated, the water pump 73 may be omitted and the high pressure air source 70 may be used instead to pressurize the probe cavity 24 and force collected water in the probe 20 through the water extraction flow conduit 31 into the collection chamber 30. In such a system, the high pressure air source 70 serves a dual function of both establishing an initial vacuum in the probe 20 to induce the inward flow of soil water and thereafter force the collected water upwards and into the collection chamber 30.

FIG. 4 is a system diagram of one embodiment of a networked date communication system 110 for establishing two-way wireless communications between one or more remote personal electronics devices and an array of sampling stations 190 using cloud computing previously mentioned above. Each sampling station 190 comprises at least one probe 20 and a sample processing sub-system 180). The “cloud” 102 acts as a communication hub which communicably and operably couples or links each personal electronic device 101 to the probe controller 60 in each of the probes 20 (see also FIG. 1 showing the probe controllers). The cloud in this situation hosts and runs appropriately configured soil analysis programs and other software (e.g. applications) configured to communicate and exchange information, operating instructions/programming, and data between each probe 20 deployed in the agriculture field and the user's personal electronic devices 101; one of which may serve as a central controller. The cloud 102 provides the hardware infrastructure (e.g. servers, databases, communication hardware, etc.) necessary to establish communication links to the probes and personal electronic devices, receive data from the probes including real-time actual soil sample analysis results and other information (e.g. pressure and temperature measurement data), store collected present and historical data transmitted by the probes in its database which can be accessed by the personal electronic devices. The probes and pedestals may communicate with the cloud 102 via cellular or satellite communication protocols, and/or local wireless/wired communications networks. Accordingly, the personal electronic devices 101 may be used to configuration probe controllers 60 of each probe 20 and control operation of the probes from a remote location near or far removed from the agriculture field where the probes are deployed.

Using all of the soil analysis data generated by the probes 20 (e.g. nutrient levels, etc.) and transmitted to the cloud system, the soil analysis software running on the cloud system in one embodiment may be configured to provide the soil water sample analysis results to the user's personal electronic device in various formats. For example, a color map of the agriculture field may be generated and presented to users on their personal electronic device display screens showing the concentration or level of the analyte of interest (e.g. nutrient levels like nitrate or other) in various regions of the agriculture field in different colors. Alternatively, the sample analysis results for the entire field may be presented in a tabular format or a combination of a color map and tabular data.

In other embodiments, each of probe controllers 60 may be operably and communicably coupled or linked to a central controller over a wireless local area network. In such a networked array of probes 20, a user's personal electronic device (i.e. cell phone, laptop, notebook, desktop, etc.) may be configured via executing a computer application or software to interact and control operation of the probes.

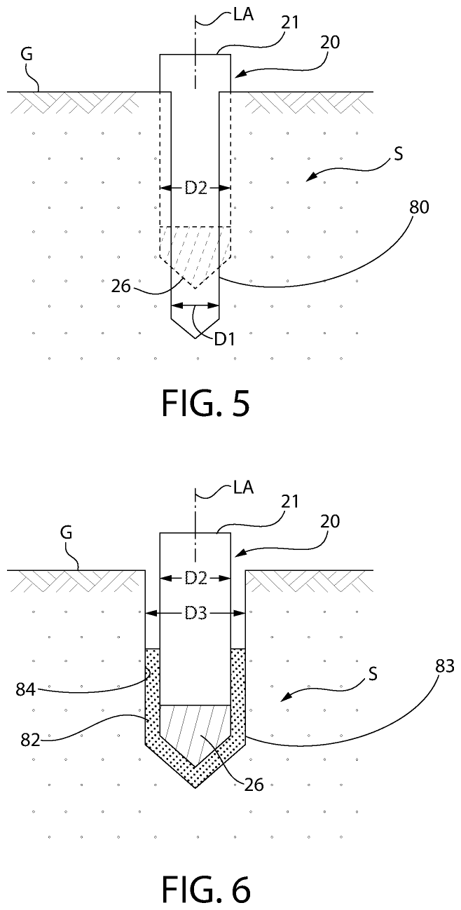

Returning now to the probes, intimate and preferably conformal contact between at least the porous media 26 of the probe 20 and surrounding soil S from which water is to be extracted for analysis is desirable to optimize drawing water into the probe. In some embodiments, the probe 20 may be placed, driven or otherwise inserted directly into virgin soil such that the probe creates its own path and opening through the soil. This ensures intimate contact with soil. In other possible embodiments shown in FIG. 5 where this is not possible or desirable either due to the density/composition of the soil and/or to protect the probe from damage during implantation, a pilot hole 80 may first be created (e.g. drilled, open via a spike or pry bar, etc.) in the soil having a diameter D1 slightly smaller than the maximum outer diameter D2 of the probe. When the probe is next vertically inserted into the pilot hole, intimate and conformal contact will be created between the probe and soil.

An alternative approach to creating intimate contact between the probe 20 and soil S is shown in FIG. 6. In this embodiment, an insertion hole 83 is formed in the soil having a diameter D3 larger than the diameter D2 of the probe. This creates an annular gap or void 84 between the probe and sides of the hole 83. This void is then filled with a hydrophilic water-absorbing media 82 (aka slush powder) having a high matric potential. Examples of suitable materials which are commercially-available include silica flour, Bentonite, super absorbent polymers (SAP) (i.e. hydrogels), or other water-absorbing materials capable of producing a capillary or wicking action conducive to draw and capture the water from the hole surrounding the probe 20. SAP (hydrogels) will form gel when exposed to water creating a highly absorbent media capable of absorbing many times it weight with water. The hydrogels absorb water through forming hydrogen bonds with the water molecules. In operation, water is drawn from the soil into the water-absorbing media which is in intimate contract with both the exposed porous media of the probe and the sides of the hole 83, and then in turn is drawn through the porous media 26 into the probe for processing and analysis.

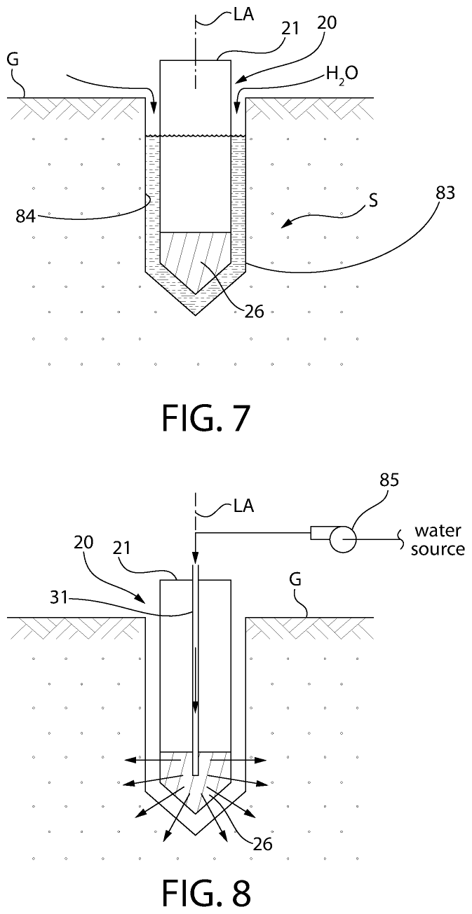

In other embodiments, the annular void 84 may instead be filled directly with water from an available source preferably upon initial installation and insertion of the probe 20 in the soil S. This is shown in FIG. 7 and creates a native soil slurry surrounding the probe. In a somewhat related embodiment shown in FIG. 8, water from a suitable in situ water source (e.g. tank, container, etc.) may be injected and back flowed through the probe and porous media 26 to mix with the soil surrounding the probe hole 83, thereby also forming a native soil slurry around the probe. In some embodiment, a pressurizing water pump 85 or other source of water pressure may be provided to back flow water through the probe for injection into the annular void 84. A separate water injection flow conduit 31 may be provided in some embodiments. It bears noting that addition or injection of water into the hole or soil surrounding the probe may also be useful for characterizing the nutrient or other properties of the soil in instances where draught-like conditions may occur resulting in insufficient amounts of natural soil pore moisture/water to allow extraction through the probe for analysis.

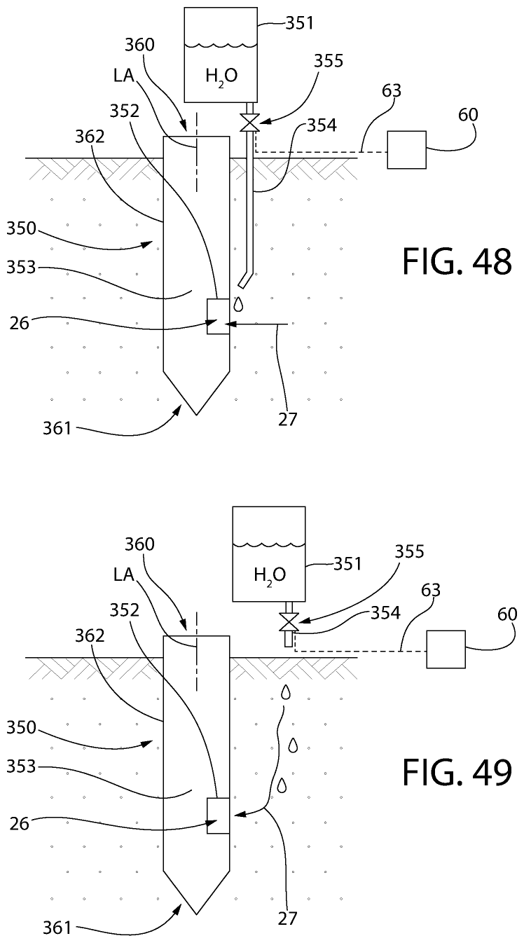

FIGS. 48-50 show additional embodiments of soil water sampling probes configured for combating the foregoing problem of insufficient naturally available soil pore water to permit sampling. Each of these figures show sampling probe systems in the form of sampling stations equipped with their own on-site source of water to facilitate artificially wetting and saturating the soil to extract a soil water sample. These self-wetting probe systems each includes a sampling probe 350 similar to probe 20 previously described herein, but further include a water system comprising a water container or tank 351 filled with sampling water to saturate and wet the soil surrounding the probe. Tank 351 preferably is located above ground as shown; but in some embodiments portions of the tank may penetrate the soil. Probes 350 each include a probe longitudinal axis LA, and elongated body defining a top end 360, bottom end 361, and circumferentially-extending sidewall 362 extending axially between the ends. An internal cavity 353 extending axially is formed between the ends. Each probe 350 includes a laterally open sampling window 352 fitted with a porous media 26 previously described herein which communicates with the open cavity 253 of the probe for applying a vacuum to the probe interior to extract water from the surrounding soil through the porous media. Any of the vacuum devices and filtrate extraction apparatuses previously described herein in conjunction with probe 20 may be used to obtain a soil water sample and transfer the sample/filtrate to the processing sub-system 180 for analysis. Each probe 350 is inserted into the soil S for a majority of its length, and substantially almost all of its length in some embodiments as shown except for its top end portion for connections to equipment enclosure 40 which houses the sample collection and analysis equipment embodied in the sample processing sub-system 180 of each sampling station 190 as previously described herein (see, e.g. FIG. 1).

Several approaches for saturating the soil in the vicinity of the sampling window 352 are disclosed. Because only the soil in the area of soil proximate to the window 352 needs to be saturated to leach or liberate the analytes (e.g. nutrients) from the soil for collection and analysis, a targeted water delivery system is preferred to minimize the volumetric capacity and size of the water tank 351 required at each sampling station.

Referring to FIG. 48, a first embodiment of a self-wetting sampling probe 350 includes a water dispensing tube 354 fluidly coupled to and extending downwards from water tank 351 alongside the exterior surface of the probe 350. In some embodiments, the dispensing tube 354 may be attached to the probe for support via suitable attachment couplings such as zip-ties, clips, etc. The bottom end of dispensing tube 354 terminates at a point proximate to sampling window 352, and preferably just above the window. The water will flow downwards a short distance through the soil by gravity to the wet the area of soil adjacent to the sampling window, which can be drawn into the probe 350 via application of vacuum to its internal cavity 353.

FIG. 49 depicts a second embodiment having the same elements as FIG. 48 described above. The dispensing tube 354 has a configuration which terminates proximate to the soil or ground surface soil-air interface, such as just above the soil/ground surface G, at the surface G, or just below the surface G. The water tank 351 and tube 354 are circumferentially arranged above the sampling window 352 so that the water runs through the soil down towards the window. In this embodiment, the water will flow downwards through the soil via gravity from a point proximate to the soil or ground surface G, and saturate the soil all the way down to at least the sampling window. The water tank 351 and dispensing tube 354 are positioned externally to the probe proximate to its external surface as in FIG. 48 discussed above.

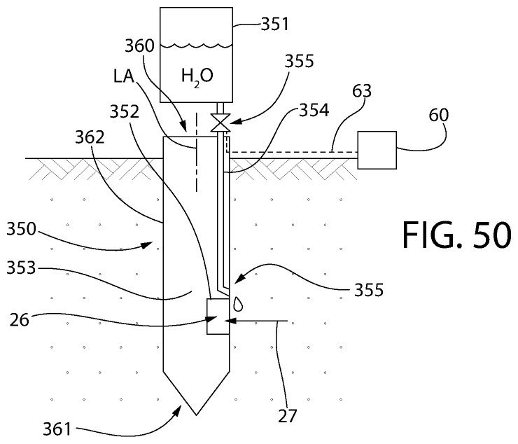

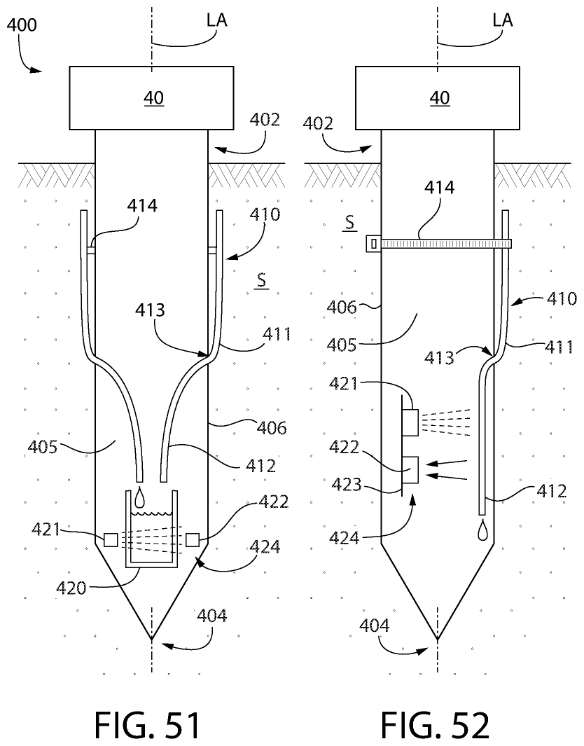

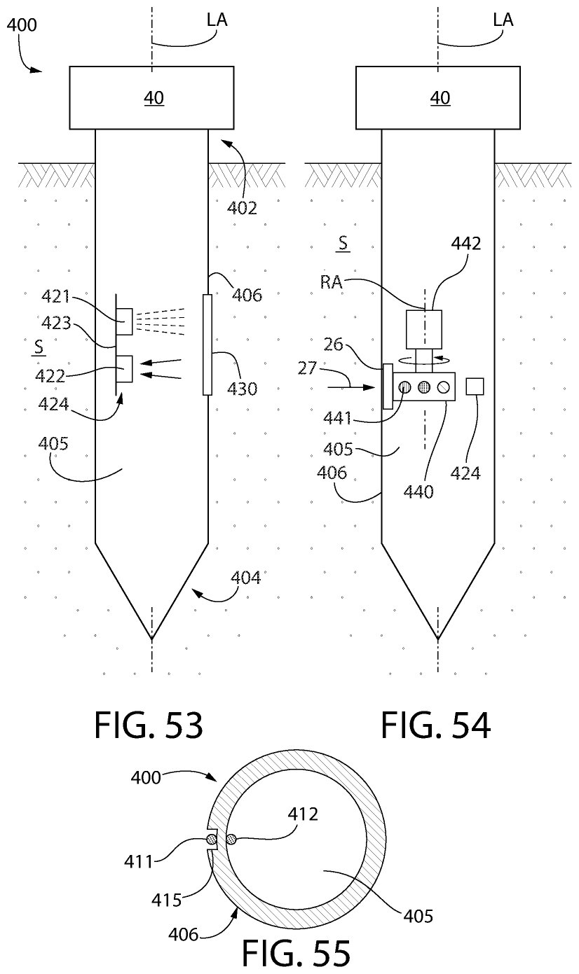

In the embodiment shown in FIG. 50, however, the dispensing tube 354 is routed internally inside the probe 350 through internal cavity 353. The bottom terminal end of the tube 354 penetrates the sidewall of the probe through a water discharge opening to dispense the water immediately above and proximate to the sampling window 352 as shown. This arrangement protects the dispensing tube 354 from the external environment. Although water tank 351 is shown immediately above and on top of the probe 350, it will be appreciated that in some embodiment the tank may be located adjacent to the probe such that the dispensing tube 354 runs laterally to and enters the cavity 353 of the probe. Either arrangement may be used.

The foregoing water dispensing tubes 354 may be made of any suitable rigid, semi-rigid, or flexible type of non-chemically reactive conduit material such as plastic or metal.

Dispensing of water from the water tanks 351 may be controlled by a control valve 355 fluidly coupled to the dispensing tube 354. Opening and closing of the valve 355 to dispense water may be automatically controlled by probe controller 60 via a communication link 63 previously described herein. Accordingly, when dry weather conditions are prevailing, the user may communicate remotely with each probe controller 60 to dispense water via opening the valve 355 for a selected duration of time sufficient to saturate the soil and obtain a water sample. In other embodiments, the flow of water may be controlled by manually operating the control valves 355.

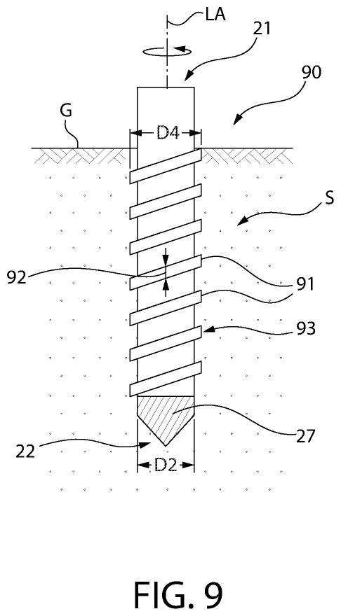

FIG. 9 shows a screw type sample probe 90 having an externally threaded body. Probe 90 includes a raised helical thread 91 which extends around the outer circumference of the probe body. Thread 91 has a larger outer diameter D4 than the diameter D2 of the cylindrical base portions of the probe. Helical thread 91 extends axially along the probe longitudinal axis LA between the top and bottom ends 21, 22 of the probe body as shown. In some embodiments, only the lower portions of the probe which will be implanted in the soil may contain the thread. Thread 91 may have raised axially elongated flat lands 93 between the valleys in some embodiments rather than sharply pointed threads to increase surface contact with and retention in the soil. The thread may terminate above the lower porous media tip 25 of the probe in some embodiments.

In lieu of linearly pushing, dropping, or otherwise inserting the threaded probe 90 into the soil, this probe may instead be rotated thereby axially/linearly translating the probe to the desired sampling depth. Threaded probe 90 advantageously eliminates the need to form a pilot or insertion hole first in the soil, and allows for easy extraction of the probe from the soil after useful service life is achieved or when terminating soil sampling. In addition, the threaded design creates greater ground engaging surface area which enhances intimate contact between the probe and soil for better induction of water into the probe. The probes stepped side profile further advantageously reduces the potential for vertical inflow of surface water in the hole surrounding the probe due to the circuitous path such surface water must take to travel downwards along the outside of the probe, thereby creating a greater resistance to flow than a straight linear path. Alternatively, in some installation, a smaller diameter pilot hole than the probe body may optionally be formed first in the soil similar to that described with respect to FIG. 5 above if desired to facilitate implantation of the threaded probe 90. This might be particularly useful for certain heavy soil types (e.g. clay).

### Hydrophilic Type Sampling Systems

According to another aspect of the disclosure, a water-absorbing hydrophilic material capable of absorbing water may be used in the water sampling probe in lieu of a vacuum to extract moisture from the soil for nutrient or other soil parameter analysis. In one embodiment, the hydrophilic material may be a hydrogel such as a superabsorbent polymer (SAP) also referred to as a “slush powder” in the art. The SAP acts as a wick which draws and moisture from the surrounding soil to the sampling probe via capillary type wicking action. The SAP may be mounted to the implanted sample probe and exposed in either direct contact with the soil, or indirectly in fluid communication with pore water in the soil via an intermediary hydrophilic fill media 82 (e.g. Bentonite, etc.) surrounding the probe in its hole such as shown in FIG. 6 and previously described herein.

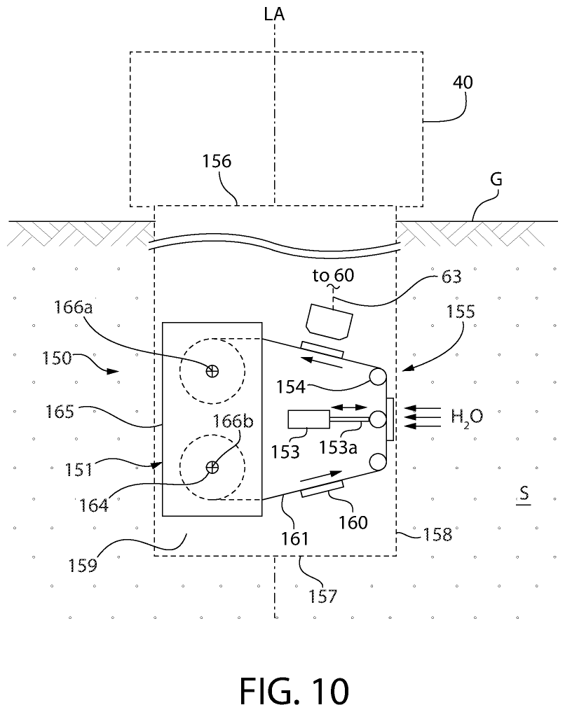

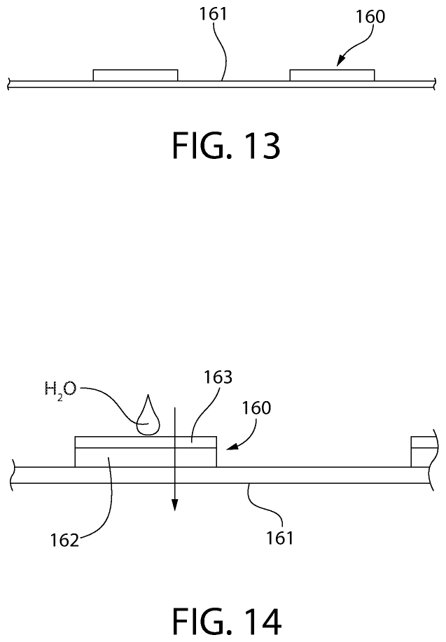

FIGS. 10-14 show a first embodiment of a hydrophilic soil water sampling system using an SAP. Referring initially to FIG. 10, the system includes water sampling probe 150 which may be similar in configuration and construction of materials to probe 20 previously described herein but without the porous medium 26. Probe 150 has a tubular body including a top end 156, bottom end 157, sidewall 158 extending between the ends, and an internal cavity 159. The tubular body may have any suitable configuration or profile such as cylindrical, rectangular cuboid, etc. with a corresponding transverse cross section.

To perform in situ chemical analysis of the soil water sample remotely at each probe 150, the SAP material may be pre-combined with one or more reagents selected to cause a detectable color change in the presence of the analyte (e.g. nutrient) of interest in the collected water, which can then be analyzed at the collection site. In one embodiment, the SAP and reagent(s) may be compressed and formed into absorbent cakes or blocks of material operable to absorb water when exposed to the soil surrounding the probe. Both the reagents and SAP may be in powdered form prior to being compressively formed under pressure into a block form.

In the present embodiment, the compressed SAP may be in the form of tests pads 160 each having a generally rigid or semi-rigid block-like structure with a composite composition formed by a combination of the hydrogel or SAP and one or more reagents. Test pads 160 each include at least a first layer 162 comprising SAP and a first reagent. In some embodiments requiring more than one reagent for a detectable color change of the water in the presence of the analyte, a second SAP and reagent layer 163 or more may be added. For example, the first reagent may be sulfanilic acid and the second reagent may be a-naphthylamine for nitrate detection and measurement. The first and second layers 162, 163 in such an embodiment may be stacked and affixed to each other by any suitable means, including being compressed together in a press to form a single multi-layer composite block or test pad 160 (best shown in FIG. 14).

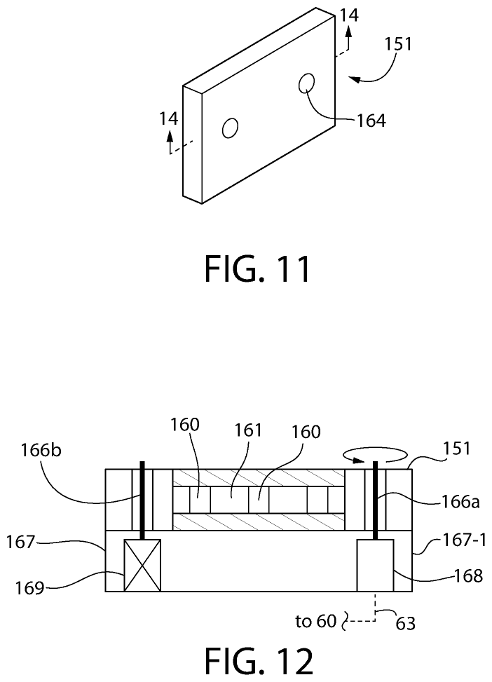

Because the probes 150 are intended to remain implanted and operable throughout of the entire growing, the probes preferably should be capable of performing multiple sample collection and analyses with minimal manual intervention and attention. In one embodiment, probe 150 may include transport mechanism configured to expose a plurality of new SAP test pads to the soil and its pore water each time a sample run is performed. In one embodiment, the transport mechanism may be a replaceable and detachable sampling cassette 151 configured for securement inside the cavity 159 of the probe 150 body (best shown in FIG. 12). With additional reference to FIGS. 11-14, cassette 151 may be similar in construction and operation to a music/video tape cassette or micro-cassette. Cassette 151 includes a rigid rectangular cuboid case 165 defining an interior space, pair of reels 164, and a movable flexible substrate wound around the reels inside the case. Other configurations of cassette cases may of course be used. The substrate may be a preferably hydrophobic polymeric sampling tape 161 as one non-limiting example; however, other flexible materials may be used. The SAP test pads 160 are rigidly affixed and coupled to the tape 161 at spaced apart increments. Test pads 160 have a thickness (measured perpendicularly to the tape) which preferably is as low in profile as possible but sufficient to draw and hold sufficient water to react properly with the reagent(s).

An electric powered tape drive 167 may be provided for operating the cassette 151 and advancing the test pads 160 in sequence for sampling. Referring particularly to FIGS. 10 and 12, tape drive 167 includes a pair of spaced apart spindles including an idler spindle 166b supported by a bearing 169 inside the drive housing 167-1 and a drive spindle 166a. Drive spindle 166a is coupled to an electric drive motor 168 of tape drive 167 which is operable to rotate the spindle for advancing the sampling tape 161 and test pads 160. Motor 168 may be located inside the drive housing 167-1 as shown. Motor 168 is operably and communicably coupled or linked to probe controller 60 via communication link 63. The probe controller 60 may thus be configured using appropriate configured software or instructions to control operation of the sampling cassette 151 including advancement of the tape and test pads for sampling and analysis of the reacted test pads after exposure to water containing the analyte (e.g. plant nutrient) of interest.

When deployed as shown in FIG. 10, a portion or loop of the substrate tape 161 may be pulled from the sampling cassette 151. The loop is guided around a pair or more of freely rotating guide spindles 154 affixed inside the internal cavity 159 of hydrophilic sampling probe 150. The guide spindles 154 are positioned to locate and expose the tape 161 and test pads 160 thereon to the soil through an open window and preferably screened sample collection window 155. The open/porous screening prevents the soil for ingress inside the probe 150 through the window. The substrate tape 161 is located proximate to window 155 so that a series of test pads 160 may be cycled to and past the window for intimate contact with the soil to absorb moisture.

To ensure intimate contact between the test pads 160 and soil through the screened window 155, probe 150 may further include a piston 153 which includes a linearly movable plunger 153a which acts on the inside facing surface of the movable tape 161 opposite the window 155. The piston 153 is operable to move the plunger 153a to engage, push, and displace the tape 161 outwards against the screen window. This places the test pad 160 on the outside facing opposite surface of the tape 161 into intimate contact with the surrounding soil to extract water therefrom (see, e.g. FIG. 10). Piston assembly 153 may be electric motor driven in one non-limiting embodiment.

Probe 150 further includes a sample analysis device 152 which may similar to device 36 previously described herein. In the present embodiment which utilizes a color change in the test pads 160 to detect the presence and concentration of the analyte, sample analysis device 150 may be a colorimeter. Analysis device 150 is preferably located proximate and adjacent to tape 161 in a position which can detect and measure the analyte is test pad 160 after it has been exposed to soil water and changed color. This may be in the return side of the tape loop as shown in FIG. 12 rather than the feed side of the loop.

Operation of the probe 150 in a process or method for capturing and analyzing a soil water sample will now be briefly described with general reference to FIGS. 10-14. The process starts with the tape 161 loop already positioned as shown in FIG. 10. However, the plunger 153a may be in a first inward retracted position either completely disengaged from or lightly engaging the tape 161. The programmable probe controller 60 advances the sampling tape 161 in a first direction via operating the drive motor 168. Tape 161 is rotated to position an unused test pad 160 adjacent to screened sampling window 155. Piston 153 is operated by the probe controller 60 to move plunger 153a outwards to an extended position. The distal end of plunger 153a abuttingly engages the sampling tape 161 forcing it and the test pad 160 outwards into intimate contact with the soil through the screened sampling window 155. The test pad 160 remains in this intimate contact position until a sufficient amount of soil water is absorbed by the SAP-reagent composition of the pad for testing. In some embodiments, probe controller 60 may activate a timer for a predetermined period of time sufficient to ensure saturation of the test pad with water from the soil. Once the timer has expired, or other means used to ensure saturation of the test pad with a water sample, plunger 153a may be retracted. The sampling tape 161 is then advanced to position the saturated test pad 160 adjacent to the colorimeter (sample analysis device 152). The color change in the test pad is analyzed by the colorimeter to measure the concentration of the analyte. The colorimeter communicates the measurement to probe controller 60 via communication link 63 (wired or wireless).

In one embodiment, the test pads 160 may be spaced apart on sampling tape 161 in intervals selected such that a new test pad is positioned adjacent to (but spaced apart from probe screened sampling window 155) and ready for the next test each time the tape is advanced to place a saturated test pad adjacent to the sample analysis device 152. In one embodiment, the piston plunger 153a may remain retracted to prevent the new pad from being saturated until the next planned soil water extraction sampling cycle. The sampling may be performed on demand and/or at regular intervals (e.g. daily, weekly, etc.) according to a sampling schedule preprogrammed into probe controller 60. When the sampling cycle is initiated by controller 60, the piston plunger is again moved to the extended position for extracting a water sample in the same manner previously described above.

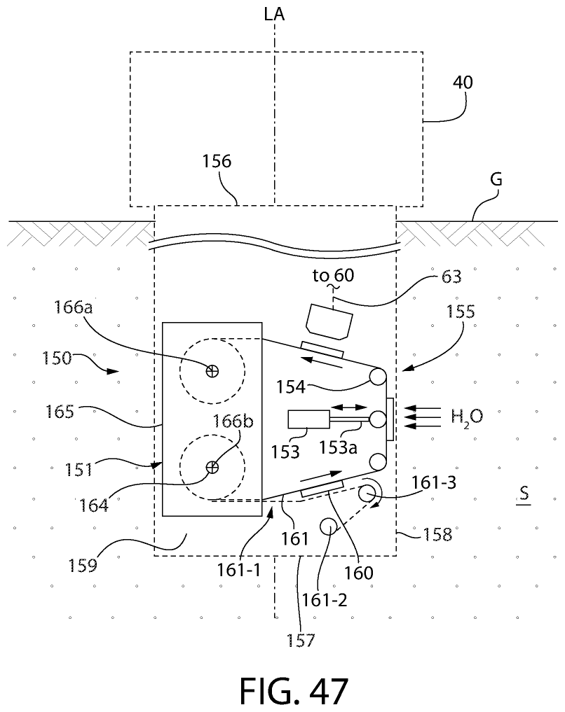

In some embodiments, a thin protective film 161-1 may be provided on sampling tape 161 which is releasably adhered to and emplaced over the test pad 160 material. This overlay film protects the test pad or strip until needed. FIG. 47 shows one example embodiment and arrangement of a powered tape drive 167 for sampling cassette 151 which is configured for use with protection film 161-1. The protective film 161-1 is automatically peeled off of the substrate tape 161 and test pads 160 by a film guide spindle 161-3 to expose the test pads just before the pads are exposed to the soil water sample which is tested. The film 161-1 is then taken up and wound on a separate used film collection wheel 161-2, while the substrate tape 161 continues along its tape path as previously described herein (see directional film path arrow and substrate tape 161 path arrows). This film 161-1 protects the sensitive testing material from oxygen and moisture while the cassette in installed in the probe beneath the soil surface.

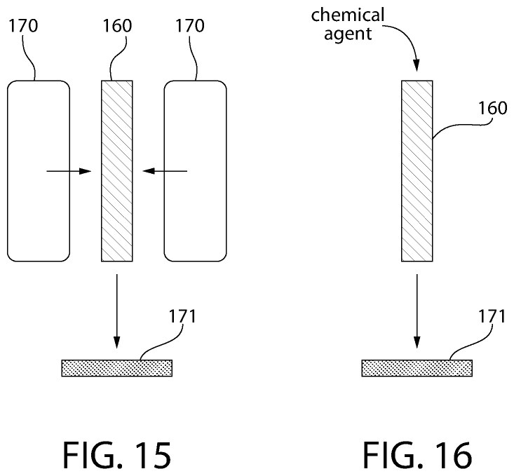

In related alternative embodiments of sampling cassette 151, pure SAP test pads 160 without reagents may be used with sampling cassette 115 in the same manner described above for simply extracting the water sample from the soil. The captured water may then be liberated from the saturated test pad for analysis of the analyte by testing means separate from the SAP (e.g. colorimeter, test strip, etc.). Such means for liberating and extracting the water captured by the hydrophilic test pads 160 may include, for example without limitation, mechanical and chemical methods. FIG. 15 shows one non-limiting example of a mechanical method includes applying mechanical pressure or force on the saturated SAP test pad 160 to release the water such as via compressing the pad between a pair of mechanical compression members 170. The captured water is released to a chemical analysis device 171 such as a test strip for analysis of the analyte. Alternatively, the release water may be collected in a container such as collection chamber 30 for further processing and analysis in the system of FIG. 1 or another. FIG. 16 shows one non-limiting example of a chemical method may be changing the pH of the saturated SAP test pad 160 by adding a chemical agent (e.g. acid or other) to break the hydrogen bonds formed between the SAP material and water molecules, thereby releasing the water to the test strip or collection container for further processing and analysis. Other approaches may be used. It bears noting that these water liberation methods and pure SAP test pads may be used without a sampling cassette 151 in some alternative sampling probes.

### Modular Soil Water Sampling Probe System

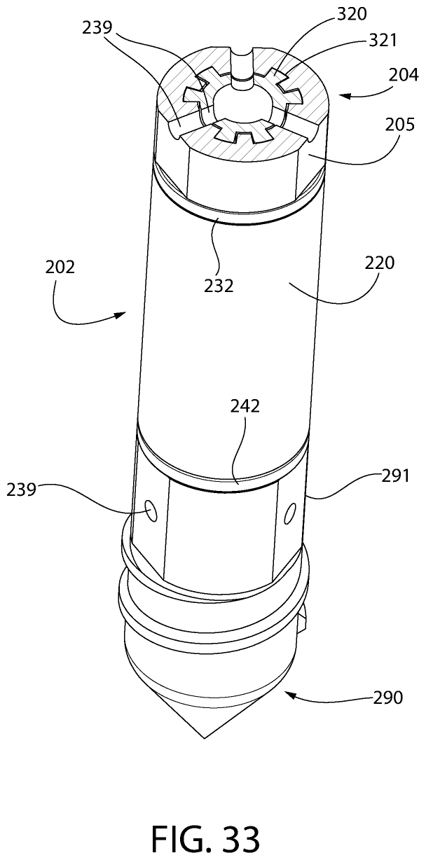

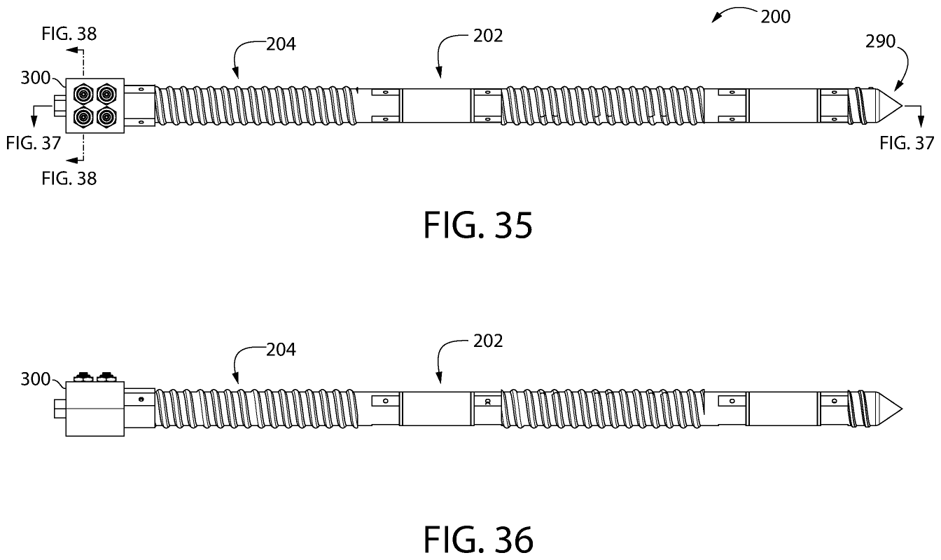

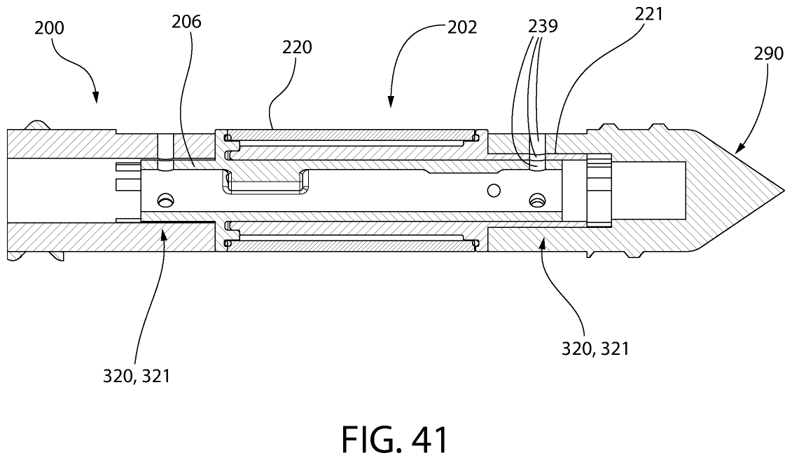

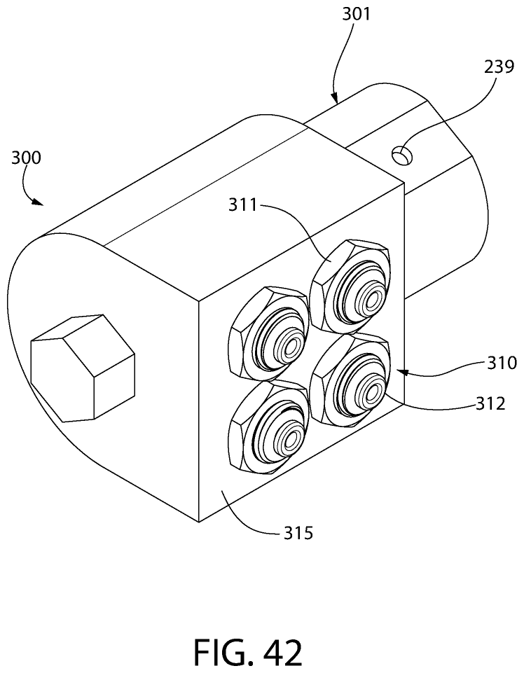

FIGS. 17-45 depict various aspects of a modular sampling probe system for extracting water from the soil for analysis and determination of analyte concentrations or levels (e.g. plant nutrients). The modular system comprises a modular sampling probe that allows the user or manufacturer to easily customize the water collection depth(s) in the soil using a plurality of interchangeable and detachably coupled components sharing a common mounting interface. Accordingly, this provides advantageously provides an expandable sampling apparatus which allows the length of the sampling probe and collection depths to be adjusted as desired. The modular sampling probe may be custom configured by the user or manufacturer via detachably coupled components to adjust the length of the probe and capture water samples at one ore multiple soil depths (simultaneously or sequentially) to generate a more complete depth profile of soil nutrient levels.

A modular sampling probe 200 for capturing soil water in one non-limiting embodiment may generally be a linearly elongated rigid structure which comprises at least one probe sampling module 202, and at least one extension member 204 detachably coupled to the sampling module by at least one coupling member 206. In some embodiments where water samples are to be collected at several elevations, a plurality of modules, extension members, and couplers may be detachably coupled together as needed. The sampling modules 202 may each be the same having the same configuration and axial length. The collection depth(s) are determined by the extension members, which may have the same configuration, but can vary in length. As an example, two sampling modules 202 may be provided in some embodiments to collect soil water samples at different locations in the soil, such as 10″ and 18″ for example. Other depths and numbers of modules may of course be used. The modules 202, extension members 204, and coupling member 206 may be detachably coupled together to allow the length of the sampling probe and water collection depth(s) to be customized in the field and/or reused and reconfigured as needed for subsequent deployments.

It bears noting that the equipment enclosure 40 which houses the sample collection and analysis equipment embodied in the sample processing sub-system 180 of sampling station 190 previously described herein (see, e.g. FIG. 1) is mounted in the agricultural field at the sampling stations in close proximity and adjacent to the modular sampling probe 200 forming an operably coupling therebetween (similarly to probe 20). The equipment enclosure 40 is not shown in FIGS. 17-45 for brevity, which depict only the modular sampling probe 200 in isolation. It will therefore be understood that the equipment enclosure 40 is included with the modular sampling probe 200 setup of the sampling station; the modular sampling probe operably coupled to sample processing sub-system 180 in enclosure 40 being substituted for the non-modular sampling probe 20.

Modular sampling probe 200 has a vertically elongated tubular body configured for implantation and embedment in the soil of an agriculture field. A spike-shaped conical end piece 290 In one embodiment, the body may be generally cylindrical in shape; however, other shapes may be used including polygonal configurations. Sampling probe 200 defines probe longitudinal axis LA and the axial direction along which the extension members 204 and sampling modules 202 are coaxially aligned and extend.

Extension members 204 may have an elongated tubular body configured for coupling to and/or between sampling modules 202. Each extension member comprises opposing coupling ends 205 of the same configuration in some embodiments, a cylindrical sidewall 208 extending longitudinally between the ends along probe longitudinal axis LA, an external surface 208, and an internal surface 201 which defines an internal passageway 203 open from end to end 205. Passageway 203 forms an interior space for internally routing vacuum and filtrate (i.e. collected soil water drawn through the filter media of the sampling modules) tubing through the unit 200 to the surface for further analysis. Passageway 203 may have a circular transverse cross-sectional shape when a cylindrical sidewall is provided as shown. Other sidewall configurations having various non-polygonal and polygonal cross-sectional shapes may also be used. In some embodiments, the sidewall 208 may be externally threaded with enlarged radial depth helical threads 209 for better soil engagement and to minimize the migration of surface water/runoff seeping downward along the outer surface of the extension members 204, which could adversely skew the measurement accuracy of actual analyte concentrations in the captured soil water sample. Threads 209 may be axially spaced relatively widely apart by flat lands 209-1 between the threads to improve gripping of and retention in the soil. The coupling end portions 205 of extension members 204 may be unthreaded.

Sampling modules 202 each comprise coupled assemblies including a porous filter 220, a filter support sleeve 221, one of the coupling members 206, and annular seals 222. Each component will be further described below.

Porous filter 220 may have an annular hollow cylindrical body defining a longitudinal internal cavity 224 extending from one end 225 to an opposing end 225. The filter body may be rigid in construction in one embodiment to withstand the application of internal vacuum pressure for extracting water samples from the soil without collapsing. Filter 220 may have an outside diameter D6 substantially equal to the outside diameter D5 of the extension members 204 so as to be substantially flush with the outer surface of the extension member as best seen in FIGS. 17 and 24 (excluding any radial protrusions of the extension member such as helical threads 209). Any suitable porous media such as those previously described herein may be used (e.g. porous ceramic, sintered metal, polymers, polymeric or fibrous membranes, screens, etc.).

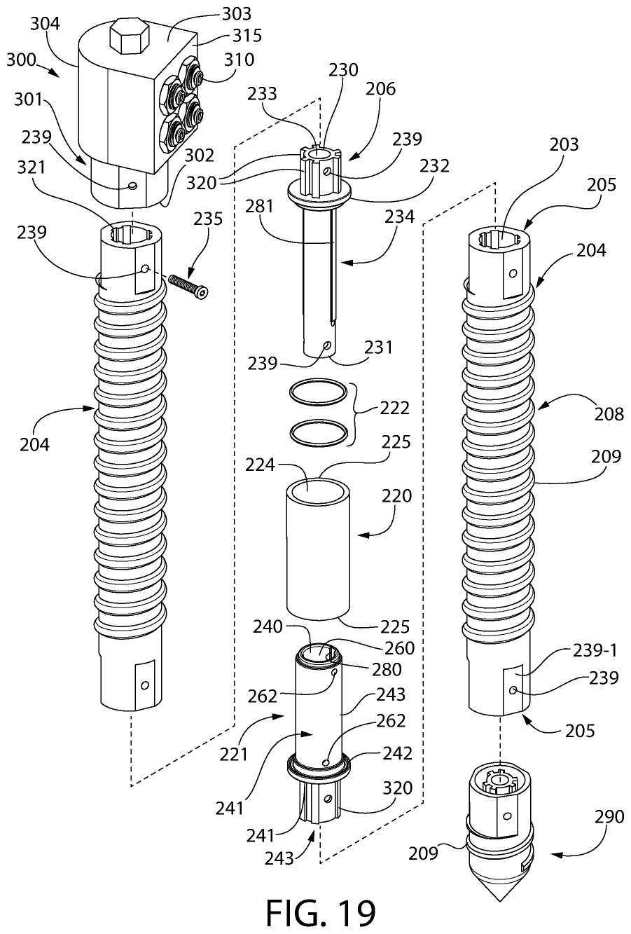

The coupling members 206 are configured for full insertion through the internal cavity 224 of the filter 220. Each coupling member 206 may have an elongated tubular and generally cylindrical body 234 comprising a first externally splined coupling end 230, opposite second plain coupling end 231, and longitudinal through passage 233 extending between and through the ends. Coupling end 230 comprises a plurality of circumferentially spaced apart external axially extending and radially projecting spline protrusions 320, thereby defining a “male” splined coupling end forming one part of the splined interface or joint previously described herein between the coupling member and a sampling module 202. Spline protrusions 320 are slideably received in complementary configured and mating internal axial spline channels 321 formed in sockets of the coupling ends 205 of extension members 204, thereby defining a “female” splined coupling end (see, e.g. FIG. 19). A splined interface and coupling is thus provided between each extension member 204 and coupling member 206. This creates a more positive and secure rotational lock between the coupling members 206 and extension members 204 which resists twisting forces when the modular sampling probe 200 is rotated and screwed into the soil rather than relying on the transverse mounting screws 235 alone which are further described below. This superior torque resistance offers by the splined interface may be a significant consideration especially when relatively long sampling probes 200 are used to extract soil water samples from relatively deep within the soil. It bears noting that the mounting fasteners 235, however, provide the important function of longitudinally locking the coupling members 206 to the extension members 204 after the spline protrusions and channels are interlocked to prevent axial separation between these parts. In alternative embodiments, the splines however may optionally be omitted. At least one pair of mating spline protrusions and channels 320, 321 are preferably provided at each splined interface. In the non-limiting illustrated embodiment, 6 pairs are shown to maximize resistance to twisting moments imparted to the sample probe assembly by resistance from the ground as the probe is rotated into the soil.

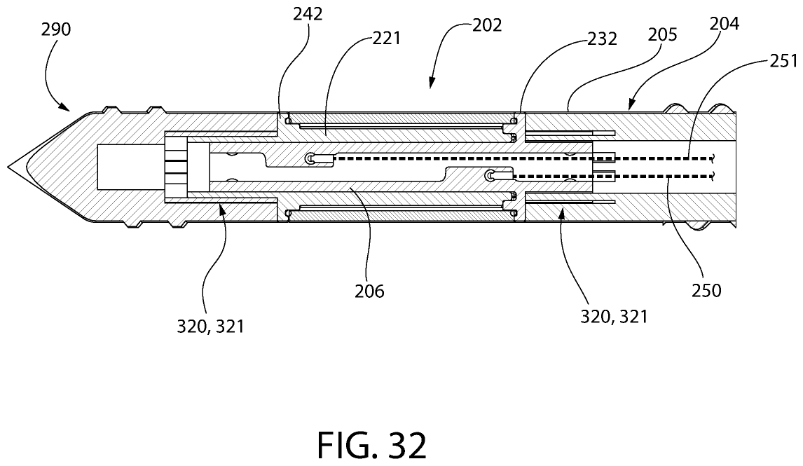

Body 234 of coupling member 206 has an axial length (measured along probe longitudinal axis LA) longer than the filter 220 and filter support sleeve 221 as best shown in FIGS. 24 and 32. This allows the coupling member to pass completely through and beyond the ends sleeve 221 and filter 220 so that the axially protruding ends 230, 231 of coupling member can be partially received inside passageway 203 of the outer extension members 204 for securement thereto. FIG. 24 shows a coupling member 206 coupled between two extension members 204 in the foregoing manner; one extension member each on opposite sides of the filter 220. FIG. 32 shows a coupling member coupled between one extension member and detachable conical end piece 290.

As noted above, the coupling ends 230, 231 of each coupling member 206 may be fastened to and longitudinally locked to corresponding coupling ends 205 of the extension members 204 by one or more transversely oriented mounting fasteners such as threaded mounting screws 235. The opposing coupling ends 205 of extension members 204 and the coupling ends 230, 231 of coupling members 206 may each include paired sets of mating radially oriented and transverse mounting holes 239 which may be rotationally and concentrically aligned with each other for inserting the threaded mounting screw 235 therethrough. The outer surface of extension members 204 may have flats 239-1 (i.e. flat surfaces) at each hole penetration location so that the heads of the mounting screws 235 can form a flat and snug abutment with the extension members when fully tightened. Holes 239 in the Mounting holes 239 may be internally threaded in some embodiments to positively engage screws 235. Other types of preferably detachable mechanical fastening mechanisms however may be used in other embodiments to longitudinal lock the coupling members and extension members together, such as for example without limitation rotatably threaded end connections between coupling members 206 and extension members 204, unthreaded pins, combinations thereof, or other type fastening means. Accordingly, the type of longitudinal locking mechanism used does not limit the invention.

With continuing general reference to FIGS. 17-45, each coupling member 206 includes an annular mounting flange 232. Flange 232 is located proximate to splined coupling end 230 but spaced inwards from the end as shown in FIGS. 19 and 24. This allows end 230 of coupling member 206 to be inserted into passageway 203 of one extension member 204. Flange 232 protrudes radially outwards from body 234 of the coupling member 206 and may have a diameter substantially equal to the outside diameter D5 of the extension members 204. This ensures smooth implantation of the modular sampling probe assembly into the soil.

The mounting flange 232 is specially configured to both engage and support one end of the filter 220 and one end of the filter support sleeve 221 of the sampling module 202 (best shown in FIG. 24). In one embodiment, the side of flange 232 facing the filter 220 includes an outer annular retention lip 237 extending circumferentially around the flange. Lip 237 abuttingly engages and seats one end of the filter. The interface between the retention lip 237 and end of filter 220 may be sealed by one of the annular seals 222. Seals 222 may be elastomeric O-rings in one embodiment.

Mounting flange 232 of coupling member 206 may further include a circumferentially-extending annular recess 236 disposed inboard of the annular retention lip 237. The recess 236 receives and engages one end 240 of the filter support sleeve 221. Recess 236 is defined by a circumferentially-extending annular filter support protrusion 238 located between the recess 26 and retention lip 237. Support protrusion 238 extends axially and perpendicularly to the flange in a direction towards the filter 220 (best shown in FIG. 24). The intersection of the protrusion 238 and the radially projecting retention lip 237 collectively form a right-angled corner or shoulder which cradles and seats one end 225 of the filter 220.

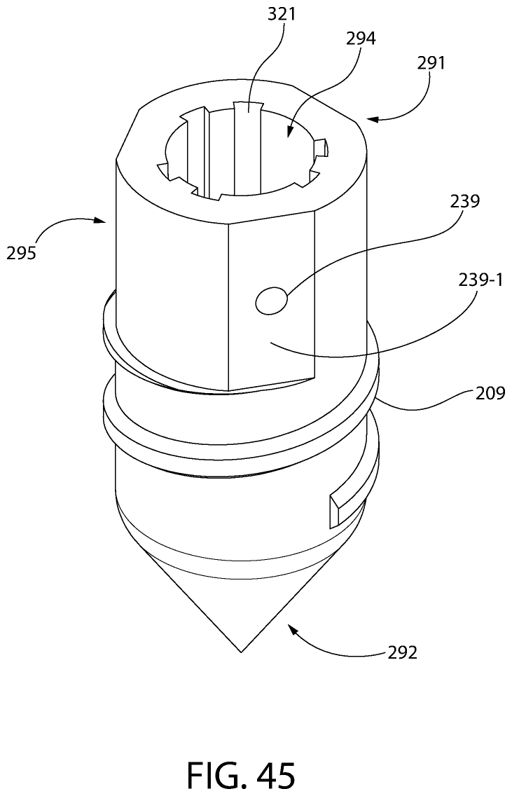

With continuing reference to FIG. 24, the filter support sleeves 221 are also configured for insertion into cavity 224 of the filter 220 from the opposite end than coupling member 206. Sleeves 221 extend for substantially the entire length of the filter 220, but do not project axially beyond its ends unlike the coupling members 206 described above. Each filter support sleeve 221 may have an elongated tubular and generally cylindrical body 243 comprising a first plain coupling end 240, opposite second externally splined coupling end 243 comprising a plurality of spline protrusions 320 previously described herein, sidewall 241, and longitudinal through passage 260 extending between and through the ends. The male splined coupling end 243 is configured to engage female spline receiving coupling end 205 with spline channels 321 of an extension member 204 (see, e.g. FIG. 19) or the conical end piece 290 version having the female spline receiving coupling end 291 with spline channels (see, e.g. FIG. 45).

Referring to FIGS. 19, 24, and 28, the inner coupling member 206 is inserted into the longitudinal through passage 260 of the outer filter support sleeve 221. A keyed joint may be provided between the coupling member and filter support sleeve. This permits relative longitudinal movement between the coupling member and filter support sleeve to allow the coupling member to slide into through passage 260 from the end 240 of the sleeve, but rotationally locks the coupling member to the filter support sleeve to prevent twisting between the sleeve and coupling member. The keyed joint may comprise a pair of diametrically opposed longitudinal keys 281 each protruding radially outwards from the external surface of the coupling member 206. Each key 281 is slideably received in one of a pair of complementary configured and diametrically opposed longitudinal keyways 280 recessed into the inner surface of the filter support sleeve 221 within through passage 260 (best shown in FIG. 28). The keys 280 and keyways 281 may extend for a majority of the axial length of the coupling member 206 and filter support sleeve 221 respectively in some embodiments for an optimum rotational lock.

Besides rotationally locking the coupling member 206 to filter support sleeve 221, the foregoing keyed joint also advantageously ensures that the fluid ports 261, 262 of the coupling member 206 and filter support sleeve 221 are automatically concentrically and radially aligned when assembled together to complete the flow passage 264 which fluidly couples the collection annulus 223 of the sampling module 202 to the vacuum and filtrates flow conduits 250, 251.

For retaining and support the end 225 of filter 220 opposite to the end engaging annular flange 232 of coupling member 206, each filter support sleeve 221 includes an annular mounting flange 242. Flange 242 protrudes radially outwards from body 243 of the sleeve and has a diameter substantially equal to outside diameter D5 of the extension members 204. Flange 242 is specially configured to both engage and support the remaining end 225 of filter 220 opposite flange 232 of the coupling member 206. In one embodiment, the side of flange 242 facing the filter 220 includes an annular outward facing retention shoulder 243 extending circumferentially around the flange. The right-angled shoulder 243 abuttingly engages and seats the remaining end of the filter as shown. The interface between the shoulder 237 and remaining end 225 of filter 220 may be sealed by another one of the annular seals 222, which may be an elastomeric O-ring.

When assembled, the filter 220 is supported and trapped between the opposing mounting flanges 232, 242 of the coupling member 206 and filter support sleeve 221 respectively. The flanges are configured to support the filter 220 in manner which is radially spaced apart from the outer surface of the filter support sleeve 221 as shown in FIG. 24. This forms a filtrate collection annulus 223 between the filter and support sleeve 221 for collecting the water sample extracted through the filter media from the surrounding soil. Annulus 223 has an axial length which extends for a majority of the length of the filter 220 (measured along longitudinal axis LA). The water sample, or alternatively “filtrate” (after passing through the filter 220), may be withdrawn directly from the collection annulus 223 by applying suction or vacuum pressure via a filtrate flow conduit as further described herein. A vacuum flow conduit may also be provided to create a negative pressure directly on the filtrate collection annulus 23 for drawing ambient soil pore water through filter 220 and into the annulus 223 for withdrawal by the filtrate flow conduit.

It bears noting that the filtrate collection annulus 223 of each sampling module 202 is fluidly isolated from ever other collection annulus to prevent mixing soil water samples collected at different elevations or depths in the soil.

As shown in FIGS. 24 and 32, the assembled sampling module 202 is a compact nested assembly comprised of the outermost external filter 220, filter support sleeve 221 therein, and innermost coupling member 206 therein. The coupling member and filter support sleeve are at least partially nested inside the filter. Accordingly, coupling member 206 has an outside diameter D2 just slightly smaller than inside diameter D1 of extension member 204 and inside diameter D3 of filter support sleeve 221 to nest inside with a minimal fit clearance to avoid excessive radial play between the parts. Diameters D1 and D3 may be substantially equal in some embodiments. Filter support sleeve 221 has an outside diameter D7 smaller than the inside diameter D4 of filter 220 by an appreciable amount necessary to form a distinct annular radial gap therebetween. The radial gap forms the water collection annulus 223 of sufficient volume to function to collect the filtrate, and allow suction or vacuum pressure to be applied to the annulus to extract water from the soil through filter 220 and to withdraw the collected resultant filtrate in the annulus to the ground surface as previously described herein. Accordingly, the radial gap which forms the collection annulus 223 is significantly larger in dimension than simply the substantially smaller and tight clearance fit gaps provided between the coupling member 206, filter support sleeve 221, and extension members 204 which simply allow those parts to be fitted/nested snugly together and would not be amenable for collecting and extracting any significant amount of filtrate for processing a sample.

In order to extract a soil water sample and transfer the filtered water (filtrate) to the surface from each sampling module 202 for processing and chemical analysis in the in situ equipment enclosure 40 with sample processing sub-system 180 therein, a plurality of flow conduits are provided. The flow conduits may include one or more suction or vacuum flow conduits 250 and filtrate flow conduits 251, which may be formed of piping or preferably small diameter tubing. Each sampling module 202 has a dedicated pair of flow conduits 250, 251; each of which are fluidly isolated form every other flow conduit to keep water samples collected at different soil depths segregated for separate processing and analysis. Vacuum flow conduits 250 may be coupled to water collection annulus 223 of each sampling module 202 at a higher elevation than the filtrate flow conduits 251 in some embodiments (see, e.g. FIG. 32) to ensure complete withdrawal of the filtrate from the annulus. The flow conduits are routed internally through the modular sampling probe 200 to the surface for direct or indirect fluid coupling to sample processing sub-system 180 components within the equipment enclosure 40 (see, e.g. FIG. 1). The vacuum flow conduit 250 may be fluidly coupled to the vacuum device (e.g. vacuum pump 32 or venturi pump 71 previously described herein). The filtrate flow conduit 251 may be fluidly coupled to the sample/filtrate transfer device which may be sample pump 73 or collection chamber 30 upstream of the vacuum pump (see, e.g. FIGS. 1 and 2). Where two or more sampling modules are provided in the modular sampling probe 200, the flow conduits 250, 251 from lower elevation sampling modules 202 are routed internally through sampling modules at higher elevations up to the equipment enclosure 40.

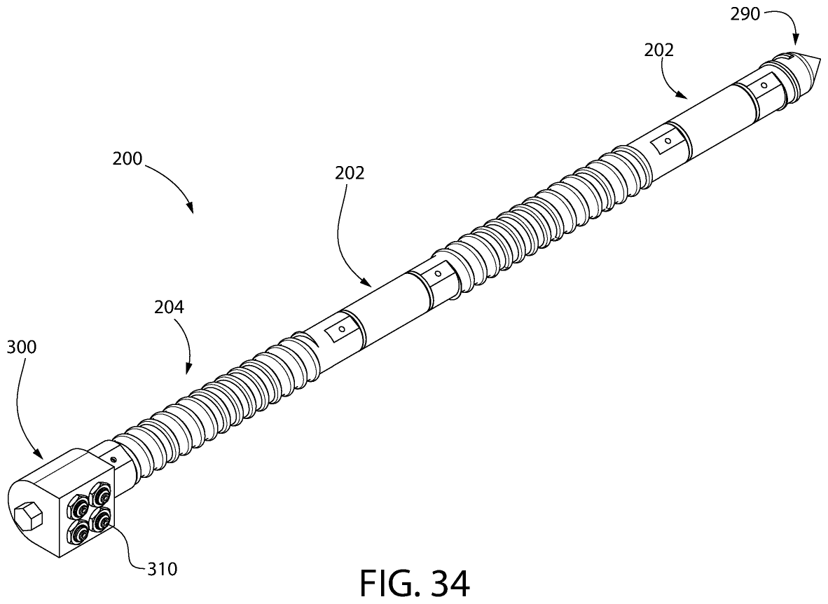

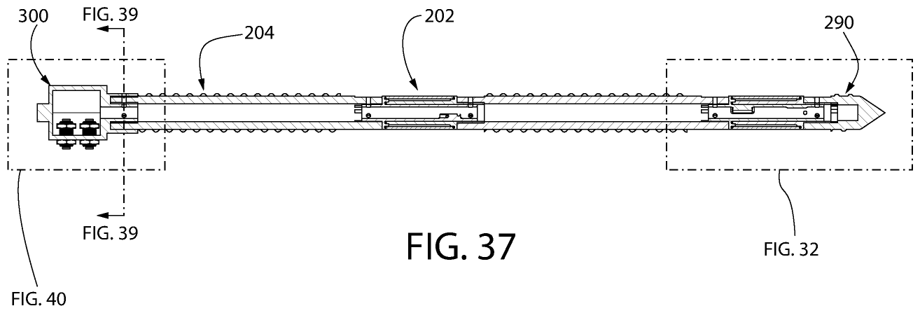

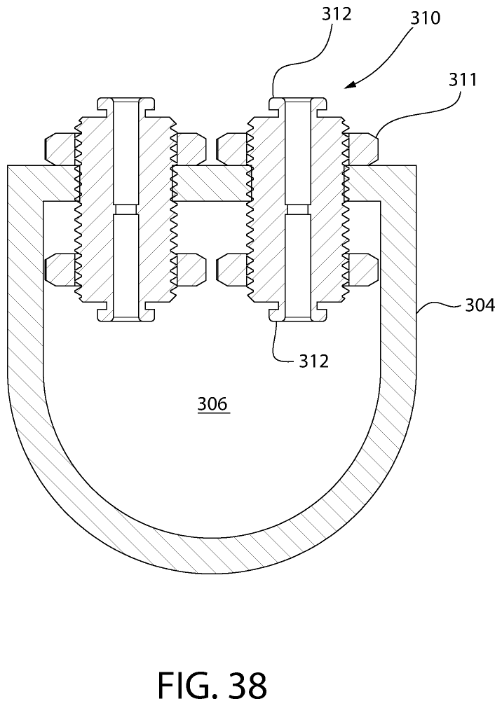

The pair of vacuum and filtrate flow conduits 250, 251 from each sampling module 202 may be fluidly coupled to a suitably configured flow manifold housing 300. In one non-limiting embodiment, the housing 300 may be configured for coupling to a coupling end 205 of the uppermost extension member 204 as shown in FIGS. 19 and 34. FIGS. 38-40 and 42-43 show the manifold housing 300 in isolation and greater detail.

Referring now to FIGS. 19, 34-40, and 42-43, manifold housing 300 has a substantially hollow box-like body comprising a top end 303, a bottom end 302, and sidewalls 304 extending between the ends which collectively define an open internal tubing chamber 206 for receiving and routing flow conduits 250, 251 therein. In one example embodiment, the body may be generally U-shaped in transverse cross section; however, any suitable shaped housing body may be used including rectangular cuboid or various other prismatic shapes.

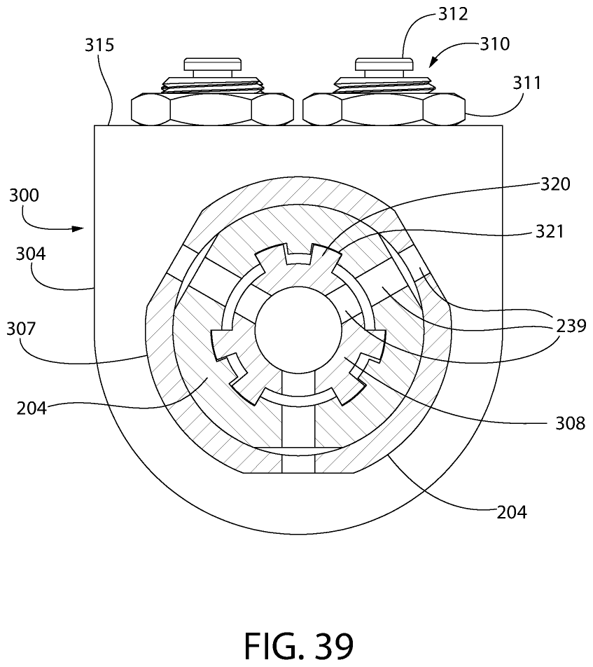

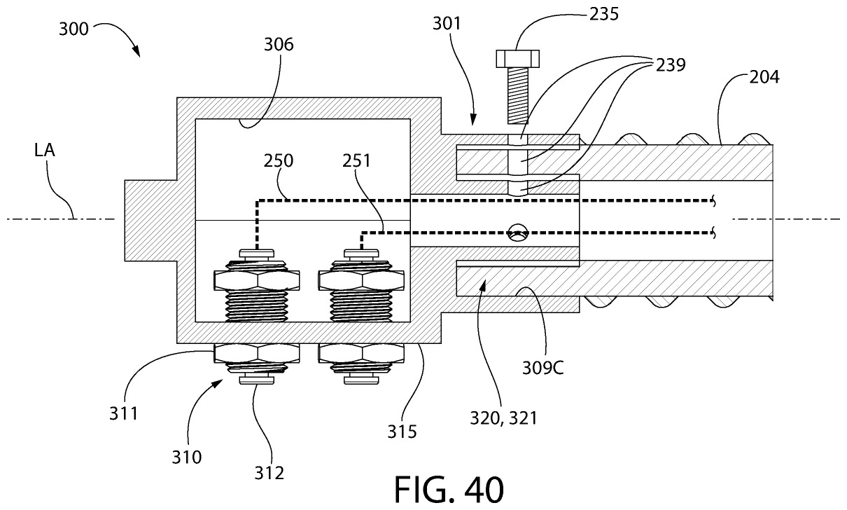

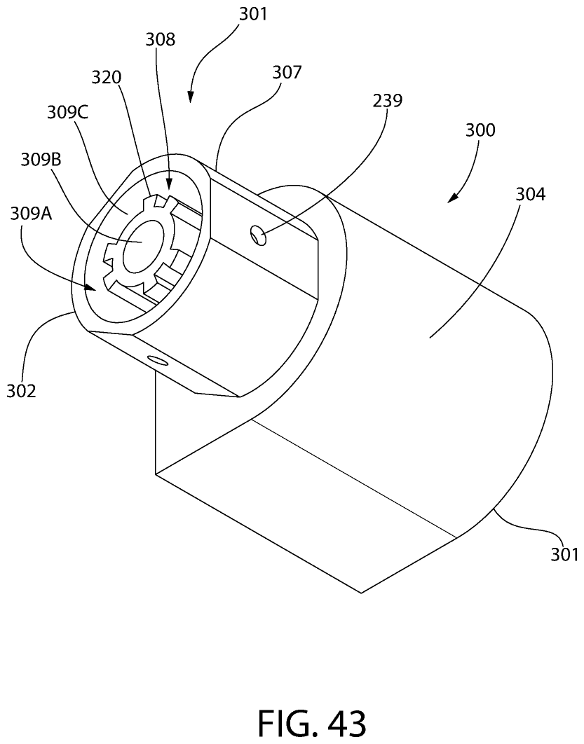

The coupling end 302 of manifold housing 300 may comprise a downwardly projecting splined coupling extension 301 configured for connection to a mating splined coupling end 205 of the uppermost extension member 204 to which the housing may be detachably secured. Coupling extension 301 has a generally double-walled tubular hollow body comprising an outer tube 307 defining an interior cavity 309A and inner splined tube 308 disposed inside the cavity (best shown in FIGS. 43). The inner splined tube 308 is concentrically aligned with the outer tube 307 and of smaller diameter forming an annular gap 309C therebetween which is configured for receiving the coupling end of the extension member 204 (see, e.g. FIGS. 39 and 40). The splined tube 308 comprises a plurality of the radial spline protrusions 320 previously described herein which are slideably received in the internal spline channels 321 of the extension member 204. This continues the same convenient common splined mounting interface used to couple the coupling members 206, filter support sleeves 221, and conical end piece 290 to the extension members. The inner splined tube 308 further defines a central passage 309B for routing the flow conduits 250, 251 into the chamber 306 of the manifold housing 300. Both the outer and inner tubes 307, 308 may include transversely open mounting holes 239 of the type previously described herein which become concentrically aligned with the similar mounting holes 239 of the extension member 204 to receive threaded mounting screw 235 therethrough for longitudinally locking the components together (see, e.g. FIGS. 39 and 40).

The manifold housing 300 further comprises a plurality of tube fittings 310. One tube fitting is provided and dedicated for each flow conduit 250, 251. The fittings 310 may have two tube connection ends 312 (one inside interior chamber 306 and one external to the housing) each configured for forming a leak resistance connection to the flow conduit tubing. The housing 300 may have a flat fitting mounting wall 315 to facilitate mounting the tube fittings 310 to the manifold housing. In one embodiment, the mounting wall 315 may be oriented parallel to the probe longitudinal axis LA such that the flow conduits make a 90 degree bend through the housing to coupling to the sample processing system 180 in the equipment housing 40 mounted proximate to the modular sampling probe 200. Other arrangement and orientations may be used. In one embodiment, the fittings 310 may straight and externally threaded. The fitting 310 extend through and project for a distance beyond the wall 315 to receive threaded nuts 311 on both the inside and outside. The nuts may be tightened from inside chamber 306 and outside to lock the tubing fittings to the manifold housing 300. The housing may be made of any suitable metallic or non-metallic (e.g. polymer) material.

In order to draw a vacuum on or collect a filtrate sample from the interior cavity 224 of filter 220, access to the filtrate collection annulus 223 from through passage 233 of coupling member 206 is needed for coupling to the flow conduits 250, 251. This requires a vacuum flow conduit penetration and a filtrate flow conduit penetration through the bodies or sidewalls of both the coupling member 206 and filter support sleeve 221 located inside filter 220. In one embodiment, coupling member 206 includes a pair of radially oriented flow ports 262 (one for vacuum, one for filtrate extraction) and filter support sleeve 221 similarly includes a pair of flow ports 262. The ports 261, 262 are arranged on the coupling member 206 and sleeve 221 such that they may be concentrically and rotationally aligned with each other to collective form a contiguous flow passageway to the collection annulus 223. The flow ports 261, 262 may be positioned to draw a vacuum and filtrate from opposite longitudinal end portions of the annulus 223.

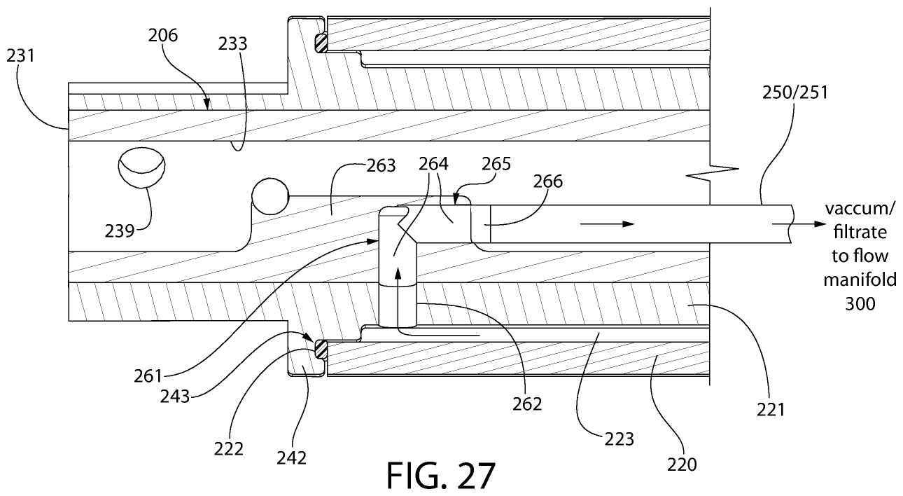

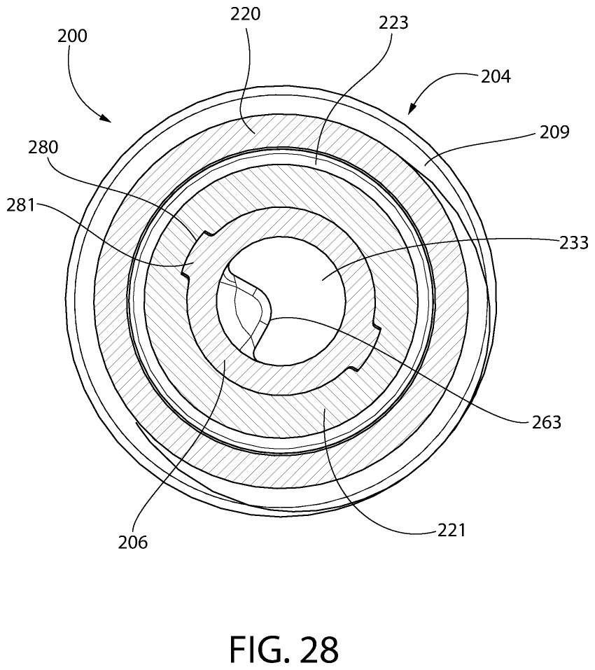

To make an angular transition from the radially oriented flow ports 261, 262 of coupling member 206 and filter support sleeve 221 (perpendicular to probe longitudinal axis LA) to the axial direction for routing the flow conduits 250, 251 upward through the modular probe unit 200 (parallel to axis LA), the coupling member 206 may include a pair of flow protrusions 263 (see, e.g. FIGS. 27 and 29-30). One flow protrusion may be used to apply a vacuum to the water collection annulus 223 for drawing soil water inwards from the soil through the filter 220 into the annulus, the other may be used for applying suction pressure to the annulus for transferring the collected filtrate to the surface for analysis in sample processing system 180. Each flow protrusion may be axially elongated and extends radially inwards from the inner surface of body 234 of coupling member 206 inside through passage 233. The extra material added by the flow protrusions 263 allows the flow ports 261 of the coupling member to form a complete internal angular flow passage 264 within each protrusion which changes direction from axial to transverse with respect to probe longitudinal axis LA. In one embodiment, as shown in FIG. 27, a 90 degree flow passage 264 may be provided to change the direction of flow from radial to axial. In such an arrangement, the flow passage 264 collectively includes a radial portion comprised of the radial flow port 261 and an axial portion 265. The flow passage 264 shown in FIG. 27 is representative in configuration of the flow passage within the other flow protrusions 263. Other angular orientations between about and including 45 degrees to 89 degrees with respect to the longitudinal axis LA may be used in other embodiments. The vacuum and filtrate flow conduits 250, 251 may be coupled directly to the flow passages 264 in some embodiments.

As best shown in FIG. 31, each of the two flow protrusions 263 in coupling member 206 is angularly and circumferentially offset from each other to simplify coupling of the flow conduit 250, 251 tubing to the protrusions and their respective flow passages 264 from the open end 230 of the coupling member (see also FIG. 32).

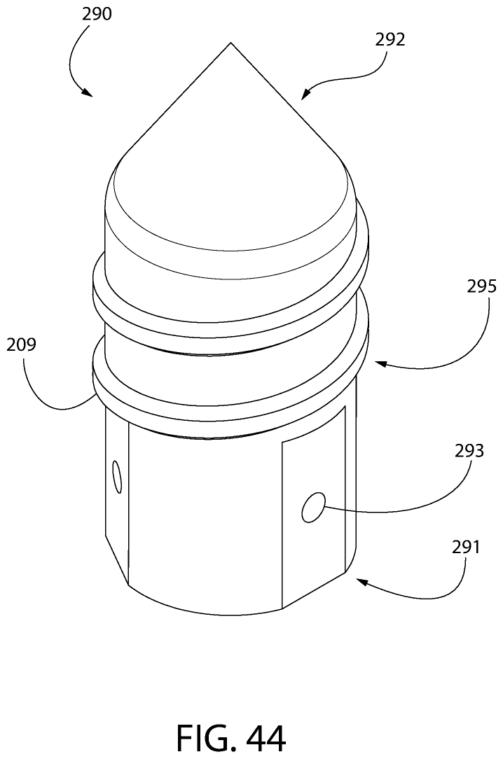

The modular sample probe 200 may have an appropriate bottom end closure to seal the bottom of the probe. One non-limiting example of an end closure is conical end piece or closure 290 as various shown in FIGS. 17-46. FIGS. 44-46 show the end closure in isolation and greater detail. End closure comprises a splined coupling end 291, opposing penetration end 292, and sidewall 295 extending therebetween. Penetration end 292 may be in the form of a spike or cone to facilitate penetrating the soil when the modular sampling probe 200 is rotated and advanced through the soil during implantation. Coupling end 291 may include the same external threads 209 used in the thread design of extension members 204 previously described herein.

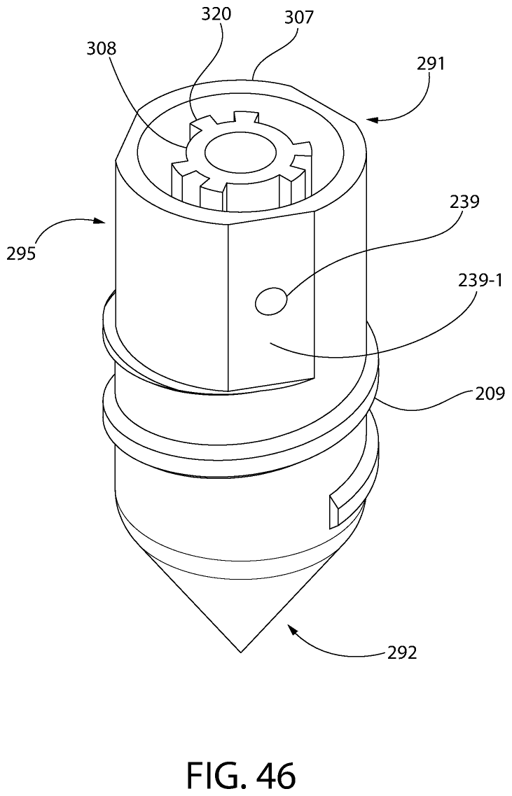

Two splined coupling end options are provided, selection of which depending upon whether the end closure 290 is to be affixed to an end of an extension member 204 as seen in FIG. 19, or to a sampling module 202 as shown in FIG. 32. FIG. 45 shows a “female” splined coupling end 291 with a socket having spline channels 321 for attachment to a “male” splined coupling end 243 having spline protrusions 320 of either a filter support sleeve 221 (see, e.g. FIG. 32) or “male” splined coupling end 230 of a coupling member 206. FIG. 46 shows an internal “male” splined coupling end 291 having the same configuration as coupling end 301 of manifold housing 300 (see, e.g. FIG. 43) for attachment to a “female” splined coupling end 205 of an extension member 204. End 205 of extension member 204 would slide inside the internal “male” splined coupling end 291 of end closure 290 to insert its internal spline protrusions 320 into spline channels 321 of the extension member in a similar manner to that shown in FIG. 40 illustrating the manifold housing 300 attachment to an extension member. End closure 290 may be formed of the same or different material as the extension members 204, which may be metallic or non-metallic as previously described herein.

It bears noting that the anti-rotational splined joints used between ends of the coupling members 206, extension members 204, filter support sleeve 221, end closure 290, and manifold housing 300 may have the male spline protrusions and the female spline channels formed on the opposite one of these components from that illustrated and described herein. Although the illustrated embodiments show one non-limiting preferred arrangement, in other arrangements the spline protrusions and channels may therefore alternatively be formed on the other opposite ones of the foregoing components in other embodiments. Accordingly, the invention is not limited by which one of the components has the spline protrusions and which one of the components has the spline-receiving channels on the ends at the joints.

A method for assembling a modular sampling probe 200 may comprise the following steps. The sub-assembly comprising the sampling module 202 may first be assembled, which in one non-limiting approach includes steps of inserting/sliding the filter support sleeve 221 into the filter 220 and inserting/sliding the coupling member 206 into the filter support sleeve as seen in FIG. 24. Alternatively, the coupling member 206 may be inserted/slid into the filter 220 first followed by inserting/sliding the filter support sleeve 221 between the coupling member and filter. Either sequence of assembly steps may be used.

In either sequence, the mounting flange 242 of the filter support sleeve 221 is abuttingly engaged with a first end 225 of filter 220, and the mounting flange 232 of coupling member 206 is abuttingly engage with a second end 225 of filter 220, thereby trapping the filter between the flanges and forming water sample collection annulus 223. Male splined coupling end 230 of coupling member 206 may then be inserted inside the socket of a female splined coupling end 205 of extension member 204 (right one shown in FIG. 24). This step comprises axially inserting the spline protrusions 320 of the coupling member 206 into the spline channels 3321 of the extension member 204. The coupling member 206 or filter assembly may be rotated to align the mating transverse mounting holes 239 in the coupling member and extension member 204. The mounting screws 235 (3 in the illustrated embodiment) may then be threaded through the concentrically aligned pairs of holes 239 to lock the coupling member 206 to the outer extension member 204.

In some embodiments, the filter 220, coupling members 206, and filter support sleeve 221 may be cooperatively dimensioned to produce tight inter-part clearances to form a friction fit between the components such that the filter assembly stays together and is self-supporting when not mounted to extension members 204. This allows the filter assemblies to be pre-assembled and stocked in order to build a customized modular sampling probe 200 when required that meets the particular sampling scenario intended.

In a broad sense with preassembled sampling modules 202 already provided and available, a method for assembling a modular sampling probe includes: providing a sampling module 202 comprising a filter arranged to come into contact with soil when embedded therein; and coupling a first end of the sampling module to a first end of an elongated extension member 204, wherein the sampling module and extension member collectively form a complete sampling probe suitable for collecting a soil water sample at a single soil depth. The coupling step may include slideably inserting spline protrusions formed on the first end of either the sampling module or the extension member into spline channels formed on the first end of the remaining other one of the sampling module or extension member.

Depending on the configuration and length of the modular sampling probe 200 being assembled, an additional extension member 204 may be secured to the remaining coupling end 231 of the coupling member 206 (left one shown in FIG. 24) in the same manner described above. Any number of extension members 204 and sampling modules 202 may be assembled in this fashion to customize the modular sampling probe 200 for the particular soil water sampling scheme intended. In the non-limiting illustrated embodiment, the modular sampling probe 200 includes two sampling modules 202 and two extension members 204 which would be operable to capture soil water samples at two different depths in the soil. It will be apparent that the length of the extension members may be selected to place the sampling modules 202 at the desired collection depths.

Once the desired probe configuration and number of sampling modules 202 are assembled, the conical end closure 290 may be coupled to the bottom of the probe 200 and the manifold housing 300 may be coupled to the top end of the probe using the splined coupling interfaces previously described herein. The vacuum and filtrate flow conduits 250, 251 may be installed and routed through the probe 200 interior and fluidly coupled by their ends to the flow protrusions 263 at bottom and at the top to the interior tube connection ends 312 of tube fittings 310 inside manifold housing 300 at a convenient time during the assembly sequence described above. Flow conduits 250, 251 are then coupled to the exterior tube connection ends 312 of fittings 310 and the sample processing system 180 fitting connections to complete fluid coupling of the sampling probe 200 to the system.

It bears noting that steps of the foregoing assembly methods may be varied in sequence in other assembly approaches to produce the completed modular sampling probe 200.

Representative materials of construction which may be used for the forgoing components of the modular sampling probe 200 are as follows. The extension members 206 and end closure 290, which do not directly come into contact with the collected filtrate, may be formed of any suitable non-metallic or metallic materials including polymers such as polypropylene, polyethylene (LDPE—low density, HDPE—high density), or machined/cast metal depending on price point and feasibility of manufacture. Preferably, the materials which come into contact with the water samples-filtrate are preferably non-corroding and chemically inert to avoid tainting the collected water samples extracted from the soil. Accordingly, the components of the sampling module 202 such as filter support sleeve 221 and coupling members 206 may be formed of injection molded polymers such as polypropylene, polyethylene, polycarbonate, etc. The internal vacuum and filtrate flow conduits may be formed of any suitable rigid, semi-rigid, or flexible non-metallic and metallic materials which are chemically insert, such as for example without limitation rigid stainless steel piping/tubing, or flexible polymeric tubing such as PTFE (polytetrafluoroethylene), FEP (fluorinated ethylene propylene), and others. The only restriction is all materials in contact with the soil or sampled water preferably should be chemically inert, therefore restricting most of the internal sampling module components and flow conduits from most metallic materials or environmentally changing materials such as nylon. It is well within the ambit of those skilled in the art to select suitable materials for the foregoing components using the criteria outlined above.

A method for operating the modular soil water sampling system will now be briefly described. In operation of the modular sampling system, a vacuum is first applied by a vacuum device to the water collection annulus 223 of the sampling module 202 via vacuum flow conduit 250, such as by starting the vacuum pump 32 (see, e.g. FIG. 1) or jet pump 71 (see, e.g. FIG. 3) in the manner previously described herein depending on which is used for the vacuum source. The pressure within the annulus will be less than the water pressure outside the probe, thereby drawing and filtering the available pore water through the filter media 220 into the annulus to collect the water sample-filtrate at this point in the process. After the vacuum device generates a vacuum on the probe, and over the course of a few hours or up to 10's of hours depending on the soil type, the pressure gradient between the soil pore water and the internal filtrate collection annulus 223 of the probe causes the pore water to travel to the area of higher hydraulic conductivity. This is important in high clay or organic matter soils, as the entrapped water and nutrients do not easily leave the soil.

After the water sample is collected and filtered, through appropriate valving and use of the manifold housing, the vacuum pump 32 or jet pump 71 can then be used apply a negative pressure or vacuum to filtrate collection annulus 223 in order to extract the filtrate for processing and analysis in the sample processing sub-system 180 in the manner previously described herein.

FIGS. 51-54 depict additional embodiments of soil water sampling probes configured for obtaining and analyzing a soil water sample for an analyte. Different approaches in each embodiment are used for collecting the sample and/or analyzing a sample. The sampling probes in each embodiment may have the same general configuration or shape, with differences in the sample collection and analysis devices as noted further below.

Referring to FIGS. 51-54, probes 400 each include a probe longitudinal axis LA, and generally elongated body defining a top end 402, bottom end 404, and circumferentially-extending sidewall 406 extending axially between the ends. The sidewall 406 may be cylindrical in shape in some embodiments with circular cross section; however, other shaped sidewalls with polygonal configurations and cross-sectional shaped may be used. Bottom end 404 may be conical to facilitate embedment in the soil S. An internal cavity 405 extending axially is formed between the ends. Each probe 350 is inserted into the soil S for a majority of its length, and substantially almost all of its length in some embodiments as shown except for its top end portion for connections to equipment enclosure 40 which houses the sample collection and analysis equipment embodied in the sample processing sub-system 180 of each sampling station 190 as previously described herein (see, e.g. FIG. 1).

FIGS. 51 and 52 depict a sampling probe 400 comprising at least one wicking member 410 for collecting a pore water sample from the soil. Wicking member 410 includes an external portion 411 and internal portion 412. External portion 411 is in direct contact with the soil S for attracting and drawing pore water from the soil via wicking capillary action. The external portion 411 may be secured to sidewall 406 by any suitable securement member 414 which may be for example without limitation clips (see, e.g. FIG. 51), cable zip ties (see, e.g. FIG. 52), or other suitable means. A plurality of securement members may be provided at axially spaced apart intervals. Preferably, in some embodiments, the securement member is selected to keep the wicking member 410 intact and in place on the exterior of the sampling probe 400 when the probe is inserted into the soil. In some embodiments, one or more longitudinally-extending external channels 415 may optionally be recessed into the sidewall 406 of the probe body for the wicking member 410 to next therein for protection against becoming dislodged when inserting the probe 400 into the soil. The channel 415 may be used in conjunction with any suitable securement members 414 (see, e.g. FIG. 55).

In one arrangement, each wicking member 410 may be oriented vertically and extends along the direction of the longitudinal axis LA of the sampling probe 400. The wicking members may enter the cavity 405 of the probe through an access aperture 413 in sidewall 406 of the sampling probe. The internal portion 412 of the wicking member may extend vertically for a length inside the probe.

In FIG. 51, a pair of wicking members 410 is shown which terminate inside cavity 405 at their bottom ends proximate to or inside a sample collection container 420. Container 420 collects soil pore water conveyed through the wicking members via capillary action which drips or is otherwise deposited in the container. The collection container may be disposed in the lower half of the sampling probe 400 in some embodiments. Any suitable shape or size container may be used. The water sample analysis device 424 for measuring the absorbance or concentration of the analyte present in the collected pore water inside container 420 may comprise UV (ultraviolet) detection in one non-limiting embodiment.

The UV detection device may comprise a UV light transmitter or emitter 421 which shines a beam of light in the UV end of the spectrum through one side of container 420, which preferably is transparent or translucent. A UV receiver or detector 422 located on the opposite side of the container measures the absorbance to ascertain the concentration of the analyte in the water sample. The UV detection device is operably and communicably coupled to programmable probe controller 60 located inside equipment enclosure 40 at the head of the sampling probe 400 (see also FIG. 1). The UV light may be in the wavelength range of 100-400 nm. The analyte of interest will dictate the wavelength of UV light used. For example, for detection of nitrate, UVC wavelength range (100-280 nm) may be used, preferably about 210-270 nm in one non-limiting embodiment for measuring either absorbance or reflectance. Other types of water sample analysis devices previously described herein may be used for measuring the analyte in the water sample. One such example is pre-priming the inside of the container with a coloring reagent (e.g. liquid or film form) which changes color of the water sample in the presence of the reagent. The absorbance or color intensity of the sample is then measured by the detection device to determine the concentration of analyte. Other examples for measuring the concentration of analyte include without limitation color measurement techniques employing single-use chemical indicating devices (test strip, exchange resin, etc.) located inside sampling probe 400 and measuring UV reflectance.

FIG. 52 shows a single wicking member 410 and a UV detection type water sample analysis device 424 comprising emitter 421 and detector 422. In this embodiment, the UV detection device is configured and arranged to measure UV reflectance directly off of the wicking member 410. A laterally broadened or flat type wick with rectangular cross-sectional shape may be used for this application to provide more laterally broadened surface area on the wick to better reflect UV light which is picked up by detector 422. Both the emitter and detector 421, 422 may be mounted on a single circuit board 423 which is located opposite the internal portion 412 of the wicking member 410 somewhere above the bottom end of the wick. The UV detection device measures the reflectance and concomitantly absorbance of the wetted wicking member 410 as it flows along and through the wicking member. Accordingly, no sample collection container is needed in this embodiment. The sample water may drip from the bottom of the wicking member and simply collect inside the sampling probe 400 at bottom. The UV detection device is operably and communicably coupled to programmable probe controller 60 located inside equipment enclosure 40 at the head of the sampling probe 400 (see also FIG. 1).

The wicking members 410 can be formed of any suitable type of wicking material and can have any suitable cross-sectional shape including polygonal or non-polygonal shape (e,g, round, rectangular, square, etc.). A membrane type wick such as those formed of hydrophilic polyethersulfone (PES) or other wicking-type membrane materials may also be used. The wicking member material selected preferably uses capillary action to transfer fluids such as the soil pore water sample. The type of wicking material selected and design of wicking member 410 may be used to control the liquid volumetric capacity and fluid conductance flow rate of the sample water collected by the wicking member. Both hydrophilic and hydrophobic type wicking materials may be used. Some non-limiting examples of suitable materials that may be used for wicking member 410 include, without limitation, cotton, felt, fiberglass, thermoplastic materials, porous plastics (e.g Porex®, polyethylene, polypropylene, etc.), porous ceramics, and synthetic fibers (e.g. rayon, polyester, viscose, etc.). The material, size, and shape of wicking member 410 does not limit the invention.

FIG. 53 shows an embodiment of sampling probe 400 which directly measures the analyte of interest in the soil via a transparent sampling window 430 located in the probe sidewall 406. A UV detection type water sample analysis device 424 comprising emitter 421 and detector 422 may be used in one implementation. Soil S is in direct contact with window 430. UV light is transmitted through window 430 to wetted soil adjoining the sampling probe 400. The reflectance is measured by detector 422 in the same manner described above to determine the concentration of analyte present in the soil. The UV detection device is operably and communicably coupled to programmable probe controller 60 located inside equipment enclosure 40 at the head of the sampling probe 400 (see also FIG. 1).

FIG. 54 shows an embodiment of sampling probe 400 which measures the analyte of interest in the soil water sample via a water sample analysis device 424 comprising a color indication carousel 440. Carousel 440 comprises a plurality of color-indicating strips or patches 441 spaced circumferentially around the body of the carousel. Patches 441 are pretreated with a color changing agent or reagent to change color when wetted by the soil pore water sample containing a specific analyte of interest (e.g. nitrate, potassium, phosphate, etc.). Carousel drive motor 442 rotates the carousel about rotational axis RA at a predetermined speed programmed into programmable probe controller 60 which forms part of a sample processing sub-system operably coupled to the carousel and water sample analysis device 424. Rotational axis RA may be vertically oriented in some embodiments as shown, however, the rotational axis may be horizontal or an angle between vertical and horizontal in other embodiments.

Analysis device 424 is configured to read and measure the color intensity of the patches 441 as the carousel 440 is continuously rotated. The color intensity is correlated to a specific concentration of the analyte. Any suitable commercially-available color detection device may be used, such as without limitation a digital colorimeter or spectrophotometer, or other device. The patches 441 carried by the carousel 440 may be wetted with the soil water sample by any of the water sample collection devices disclosed herein (see, e.g. FIGS. 47-52) which are configured to direct the collected pore water onto the patches. In the non-limiting illustrated example, a porous filter or media 26 is shown which is in direct contact with the soil through an opening in the side of sampling probe 400 (e.g. sidewall 406). The media 26 may be made of a wicking material such as those described above which draws and absorbs pore water in the soil (see directional water flow arrow 27). The carousel 440 may be in direct contact with the media 26 such that the color-indicating patches 441 are wetted by water in media 26 as the carousel rotates. The porous media 26 in one may be one side of the carousel as shown, and the color detection analysis device 424 may be on another side (either adjacent or opposite to the device.

While the foregoing description and drawings represent some example systems, it will be understood that various additions, modifications and substitutions may be made therein without departing from the spirit and scope and range of equivalents of the accompanying claims. In particular, it will be clear to those skilled in the art that the present invention may be embodied in other forms, structures, arrangements, proportions, sizes, and with other elements, materials, and components, without departing from the spirit or essential characteristics thereof. In addition, numerous variations in the methods/processes described herein may be made. One skilled in the art will further appreciate that the invention may be used with many modifications of structure, arrangement, proportions, sizes, materials, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being defined by the appended claims and equivalents thereof, and not limited to the foregoing description or embodiments. Rather, the appended claims should be construed broadly, to include other variants and embodiments of the invention, which may be made by those skilled in the art without departing from the scope and range of equivalents of the invention.

1. A system for collecting and analyzing soil water samples comprising:
a sample probe comprising a filter media arranged to contact the soil when embedded therein, the sample probe configured for collecting a water sample from the soil; and
a sample processing sub-system located proximately to the sample probe, the processing system operably coupled to the sample probe and configured to extract and analyze the water sample for at least one analyte.

2. The system according to claim 1, wherein the sample processing sub-system includes a processor-based probe controller, the probe controller configured to direct operation of the sub-system.

3. The system according to claim 2, wherein the probe controller is configured to communicate with a remote electronic device defining a central controller.

4. The system according to claim 2, wherein the sample processing sub-system includes a vacuum device configured for generating a vacuum in the sample probe to extract a water sample from the soil, and a water sample analysis device configured for measuring a concentration of an analyte in the water sample.

5. The system according to claim 4, wherein the water sample analysis device is selected from the group consisting of a colorimeter, ion selective electrodes, and ion exchange resins.

6. The system according to claim 1, further comprising a tape for collecting the sample.

7. The system according to claim 6, wherein the tape further comprises a test pad which collects the sample.

8. The system according to claim 7, further comprising a removable film covering at least the test pad.

9. The system according to claim 6, wherein the tape is movably disposed in a cassette.

10. The system according to claim 9, wherein the cassette comprises a tape drive mechanism operable to dispense the tape.

11. The system according to claim 10, wherein the tape is wound around an idler spindle and a drive spindle operably coupled to the drive mechanism, the drive mechanism operable to feed a continuous length of the tape from the drive spindle to the idler spindle.

12. The system according to claim 9, wherein an active testing portion of the tape is movably guided around a pair a guide spindles arranged inside the sampling probe when the drive mechanism is operated.

13. The system according to claim 12, wherein the active testing portion lies external to the cassette and is positioned for exposure to the soil through a window of the sampling probe.

14. The system according to claim 6, further comprising a piston configured to move the tape into a window in the sample probe.

15. The system according to claim 6, further comprising a colorimeter disposed to analyze the soil sample on the tape.

16. The system according to any preceding claim 1, further comprising
a water system comprising a tank containing sampling water located proximate to the sample probe, the water system configured to wet the soil surrounding the probe.

17. The system according to claim 16, wherein the water system comprises a dispensing tube fluidly coupled to the tank, the dispensing tube configured to dispense the sampling water in a vicinity of the soil adjacent to the filter media.

18. The system according to claim 17, wherein the dispensing tube is arranged external to the probe.

19. The system according to claim 17, wherein the dispensing tube is arranged inside the probe. 20, (Currently Amended) A modular sampling probe for collecting soil water samples comprising:
at least one sampling module comprising a filter arranged to contact the soil when embedded therein, the sampling module configured and operable to extract and filter a water sample from the soil; and
at least one extension member coupled to the at least one sampling module at a first joint.
a sampling probe configured for embedment in soil;
at least one wicking member including a first portion arranged to contact the soil and capture a water sample, and a second portion inside the probe, the wicking member formed of a wicking material structured to extract and transport the captured water from the soil via capillary action; and
a water sample analysis device configured to measure an analyte present in the water sample captured by the wicking member .

21. A system for collecting and analyzing a soil water sample comprising:
a sampling probe configured for embedment in soil;
at least one wicking member including a first portion arranged to contact the soil and capture a water sample, and a second portion inside the probe, the wicking member formed of a wicking material structured to extract and transport the captured water from the soil via capillary action; and
a water sample analysis device configured to measure an analyte present in the water sample captured by the wicking member.

22. A system for collecting and analyzing a soil water sample comprising:
a sampling probe configured for embedment in soil;
a sampling window disposed on sampling probe and in direct contact with the soil outside of the sampling probe; and
a water sample analysis device configured to emit ultraviolet light at the soil through the sampling window and measure reflectance of the ultraviolet light from the soil to measure an analyte present in pore water of the soil.

23. A system for collecting and analyzing a soil water sample comprising:
a sampling probe configured for embedment in soil;
a rotatable carousel disposed inside the sampling probe, the carousel comprising a plurality of circumferentially arranged color-indicating patches;
a sample collection device configured and arranged to collect the water sample from the soil and wet the color-indicating patches;
wherein the patches are operable to change color in the presence of an analyte in the soil water sample.

24.-60. (canceled)
